(19) United States
(12) Patent Application Publication
(10) Pub. No.: US 2023/0272493 A1
(43) Pub. Date: Aug. 31, 2023

(54) VIRAL AND HOST BIOMARKERS FOR DETECTION, THERAPEUTIC EFFECTIVENESS, AND MONITORING OF CANCER LINKED TO SARS-CoV-2 AND HUMAN PAPILLOMA VIRUS

(71) Applicant: Rafael Guerrero-Preston, Baltimore, MD (US)

(72) Inventor: Rafael Guerrero-Preston, Baltimore, MD (US)

(21) Appl. No.: 18/135,484

(22) Filed: Apr. 17, 2023

Related U.S. Application Data
(63) Continuation-in-part of application No. PCT/US2021/010031, filed on Aug. 4, 2021.

(51) Int. Cl. C12Q 1/70; C12Q 1/6886

(52) U.S. Cl. CPC C12Q 1/708; C12Q 1/701; C12Q 1/6886; C12Q 2600/158; C12Q 2600/154; C12Q 1/6844

(57) ABSTRACT

Methods of detecting, including early detection, of cancer mediated by SARS-CoV-2 and HPV, may include detection of such viral infections alone or together with one or more biomarkers selected from gene specific DNA methylation levels, whole genome DNA methylation levels, host RNA expression levels, T-Cell receptor amount or clonality, B-Cell receptor amount or clonality, microbiome. One method includes analysis of SARS-CoV-2 nucleic acid and the one or more biomarkers from samples of the same tissue, biofluid, or both. These methods are useful for, among other things, assessing the effectiveness of treatment, monitoring relapse, and clinical staging of cancer. These methods are also useful for among other things to monitor the effectiveness of strategies and therapies used to modify lifestyle and contextual effects to prevent disease, foster wellness and enable health promotion.

### CROSS-REFERENCE TO RELATED APPLICATIONS

This application is a continuation-in-part of co-pending international application PCT/US2021/010031, filed Aug. 4, 2021, which designates the United States, the contents of which are hereby incorporated by reference in their entirety.

### STATEMENT REGARDING FEDERALLY SPONSORED RESEARCH

This invention was made with government support under grant number 5R44 MD014911-03 granted by the National Institute of Minority Health and Health Disparities. The government has certain rights in this invention.

### TECHNOLOGY

This invention relates to diagnostic, screening, and early detection methods for oncogenic Human Papilloma Virus (HPV) mediated tumors, such as cervical, oropharyngeal, anal, and penile cancer, in patients co-infected with SARS-CoV-2, which can also be used to monitor therapeutic effectiveness of treatments and relapse monitoring.

### BACKGROUND

The coronavirus disease 2019 (COVID-19), an infection caused by severe acute respiratory syndrome coronavirus 2 (SARS-CoV-2) is the first global pandemic of the 21^{st} century. Several genomic epidemiology tools have been developed to track the public and population health impact of SARS-CoV-2 community spread worldwide.

As of April 2023, the WHO has tracked more than 762,201,000 confirmed cases of COVID-19, including 6.893,190 deaths, since the beginning of the pandemic in January 2020. A SARS-CoV-2 Variant of Concern (VOC), known as 201/501Y.V1, VOC 202012/01, or B.1.1.7, was detected in the United Kingdom in November 2020 and has now spread to multiple countries worldwide. Genomic epidemiology studies reveal B.1.1.7 possesses many non-synonymous substitutions of biological/immunological significance. in particular Spike mutations HVA69-70, N501Y and P681H, as well as ORF8 Q27stop and ORF7a. B.1.1.7 shows increased transmissibility and has rapidly become the dominant VOC in the United States (US) (https://covid.cdc.gov).

The HVA69-70 mutation is a deletion in the SARS-CoV-2 21765-21770 genome region that removes Spike amino acids 69 and 70. The HVA69-70 causes target failure in the TaqPath COVID-19 RT-PCR Combo Kit (ThermoFisher) assay, catalog number A47814 (TaqPath). TaqPath is designed to co-amplify sections of three SARS-CoV-2 viral genes: Nucleocapsid (N); Open Reading Frame lab (ORF lab)′, and Spike (S). The Spike HVA69/70 deletion prevents the oligonucleotide probe from binding its target sequence, leading to what has been termed S gene dropout or S gene target failure (SGTF). SGTF is associated with significantly higher viral loads in samples tested by TaqPath. S gene target late amplification (SGTL) has also been observed in a subset of samples having cycle threshold values for S gene >5 units higher than the maximum Ct value obtained for the other two assay targets: N and ORF lab.

The US and countries where B.1.1.7 rapidly became the dominant SARS-CoV-2 variant require immediate and decisive action to minimize COVID-19 morbidity and mortality. However, the US does not have a national genomic epidemiology surveillance network for COVID-19 whole genome sequencing (WGS) program in place. Therefore, only a small fraction of all new cases is being sequenced ad-hoc. SGTF has been shown to correlate with the A69-70 mutation highly. Evidently, SGTF can be used as a proxy to monitor SARS-CoV-2 lineage prevalence and geo-temporal distribution and may be near-direct measure of B.1.1.7.

SARS-CoV-2 invades target cells by binding to angiotensin-converting enzyme (ACE) 2 and modulates the expression of ACE2 in host cells. ACE2, a pivotal component of the renin-angiotensin system, exerts its physiological functions by modulating the levels of angiotensin II (Ang n) and Ang-(1-7). ACE2 is widely expressed in the ovary, uterus, vagina, and placenta. Therefore, apart from droplets and contact transmission, the possibility of mother-to-child and sexual transmission also exists. To date, COVID-19 has not been reported to be sexually transmitted.

Loss of ACE2 promoter DNA methylation linked to ACE2 upregulation has been observed in colon adenocarcinoma, kidney renal papillary cell carcinoma, pancreatic adenocarcinoma. rectum adenocarcinoma, stomach adenocarcinoma, and lung adenocarcinoma. DNA methylation dysregulation may also facilitate viral entry. viremia. and an excessive immune response to SARS-CoV-2.

Widespread testing of asymptomatic people is critical to identify infected individuals and help inform individual quarantine efforts and overall management guidelines for such highly infectious and long-shedding viruses such as SARS-CoV-2. particularly among asymptomatic residents of Black and Latino communities, who are most susceptible and at high risk for SARS-CoV-2 morbidity and mortality. Although nucleic acid-based tests can reveal the presence of the virus, the host epigenome response can provide needed unique information about host and SARS-CoV-2 dynamics. Epigenome modulation by SARS-CoV-2 infection is bound to impact infectivity, morbidity, and mortality trends in infected individuals. Molecular testing is a rapid means to determine if someone has been exposed to SARS-CoV-2, and also plays an important stratification for patients that are less likely to have any virus being shed, and thus likely less contagious or not a risk at all.

However, SARS-CoV-2 testing services are not yet being offered in cervical cancer screening clinics. Consequently. we do not have incidence or prevalence data of SARS-CoV-2 infection among asymptomatic women who are seen in cervical cancer clinics in the US. We argue that the approximately 40 million women who annually receive a PAP test result in the US, should be co-tested for SARS-CoV-2 in order to identify asymptomatic COVID-19 patients. This opportunity will also allow us to study if host DNA methylation markers of cervical dysplasia are modulated by SARS-CoV2 infection in HPV+ women.

The stability of our genome and correct gene expression is maintained to a great extent thanks to a perfectly preestablished pattern of DNA methylation and histone modifications. In cancer and other chronic diseases this scenario breaks down due a sudden loss of global methylation associated with histone modifications which lead to genomic instability. chromosomal rearrangements. activation of transposable elements and retroviruses, microsatellite instability and aberrant gene expression. In cancer an interesting gene-specific phenomenon following global DNA hypomethylation has been widely studied whereby the regulatory regions (CpG islands) of certain tumor suppressor genes (such as BRCA1, hMLH1, p16^{INK4a}, and VHL) become hypermethylated, inactivating the gene as a consequence, whilst the regulatory regions of proto-oncogenes become hypomethylated thus leading to transcriptional activation of the oncogene. Thus, global DNA hypomethylation is usually seen together with gene-specific hyper and hypomethylation in cancer and other chronic diseases. The global methylcytosine content of a large collection of normal tissues and tumors has been studied to begin to understand this mechanism in cancer and other diseases.

The human epigenome is dynamic, not only throughout the cell cycle and during mitotic divisions, but also in its response to environmental factors, which can be critical in development and during aging. Transient and fixed epigenetic modifications continually modulate the normal human epigenome throughout the life course in response to endogenous and exogenous stimuli. The epigenome serves as an interface between the dynamic environment and the inherited static genome. configured during development to shape the diversity of gene expression programs in the different cell types of the organism by a highly organized process. It has been shown that exposure to physical, biological, and chemical factors, as well as exposure to social behavior, such as maternal care, modifies the epigenome. Therefore, exposures to different environmental agents throughout the life course may lead to interindividual phenotypic diversity, as well as differential susceptibility to disease and behavioral pathologies.

The responses of the epigenome to environmental exposures throughout the life-course are not just aberrations leading to pathology but a biological mechanism that serves as a medium for the adaptability of the genome to altered environments during life. External exposures, physical, chemical, biological, and physical exposures received at different levels of social organization lead to changes in the extracellular environment of developing or mature somatic cells. activating signaling pathways, which link extracellular environmental exposures and epigenetic machineries.

The epigenomic machineries are the biological substrate that serves as a mediator between endogenous and exogenous stimuli at different levels of biological organization and the resultant gene expression, which leads to adaptive or reactive responses to said stimuli. The interaction between the internal or external environment and the epigenome is exposure, tissue, and cell specific. Therefore, environmental stimuli lead to changes in gene expression levels by interacting with epigenetic machineries without altering the sequence of DNA bases. This interaction leads to a modulation in biological and/or psychological processes that modulate gene expression, in transient and permanent fashion through-out the life-course: from womb to grave. The interaction between non-genotoxic environmental stressors and environmental health promoters and the epigenome occurs at different pathways and intersections of cellular. organ, systemic and bodily functions; from memory formation and synaptic plasticity to adaptation to changing environments.

DNA methylation, the most important epigenetic modification known, is a chemical modification of the DNA molecule itself, which is carried out by an enzyme called DNA methyltransferase. DNA methylation can directly switch off gene expression by preventing transcription factors binding to promoters.

Cancer is one of the leading causes of morbidity and mortality worldwide, and early detection of cancer is critical for effective treatment and better prognosis. Human papillomavirus (HPV) is known to be associated with various types of tumors, including cervical, anal, penile, and oropharyngeal cancers. In the context of the SARS-CoV-2 pandemic and the emergence of Long COVID, there is a need for innovative diagnostic techniques that can facilitate early cancer detection and follow-up of HPV-related tumors in this patient population without causing discomfort or further complications.

Currently, invasive methods such as biopsy and imaging techniques are used for cancer detection and follow-up. However. these methods are not suitable for all patients, particularly those with SARS-CoV-2 infections or Long COVID. Consequently. there is a need for non-invasive, cost-effective, and accurate diagnostic methods for this patient population.

### SUMMARY

In one aspect, a method for detection of cancer risk mediated by SARS-CoV-2 and oncogenic Human Papilloma Virus (HPV) includes detecting nucleic acids in a subject at risk of cancer linked to HPV infection. The method may include detecting and quantifying SARS-CoV-2 nucleic acid in a sample collected from a subject. The sample may comprise tissue or body fluid. The method may further include comparing the amount of SARS-CoV-2 nucleic acids in the sample to the presence of HPV DNA or RNA. whereby if the value of SARS-CoV-2 nucleic acids in the sample is high in HPV positive subjects, then the subject has an increased risk of accelerated pre-malignant progression, development of cancer, having cancer, or cancer progression.

In one example, the method includes collecting and/or isolating the sample from a subject.

In one example, the sample is a biospecimen selected from one or more biofluids, one or more tissues, or combination thereof.

In the above or another example the sample includes RNA, and the method includes using a reverse transcription process to convert the RNA into cDNA. In a further example, quantifying the SARS-CoV-2 nucleic acids includes using Real-Time Polymerase Chain Reaction (RT-PCR), Loop Mediated Amplification (LAMP), or SARS-CoV-2 Whole Genome Sequencing (WGS).

In any of the above examples or another example, the sample is a cervical liquid cytology sample, saliva sample, urine sample, cervical smear, vaginal lavage fluid sample, anal smear, stool sample, tumor sample, tissue sample, or any combination thereof.

In any of the above examples or another example, the cancer is one of oral cancer, tongue cancer, oropharyngeal cancer, anal cancer, penile cancer, vulvar cancer, or vaginal cancer.

In any of the above examples or another example, the method further includes quantifying human (host) genes using Real-Time Polymerase Chain Reaction (RT-PCR). Loop Mediated Amplification (LAMP), or SARS-CoV-2 Whole Genome Sequencing (WGS).

In any of the above examples or another example, the method includes comparing the levels of SARS-CoV-2 gene expression in the sample to the presence of HPV DNA or RNA, whereby if the expression value of SARS-CoV-2 genes in the sample is high in HPV positive subjects, then the subject has an increased risk of accelerated pre-malignant progression, development of cancer, having cancer, or cancer progression.

In any of the above examples or another example, the method includes comparing gene expression of human (host) genes to the presence of SARS-CoV-2 and/or HPV DNA or RNA, whereby if the expression value of human genes in the sample correlates (positively or negatively) in HPV and SARS-CoV-2 positive subjects, then the subject has an increased risk of accelerated pre-malignant progression. development of cancer, having cancer, or cancer progression.

In any of the above examples or another example wherein the sample or an additional sample collected from the subject includes genomic DNA. In a further embodiment, the method includes isolating the sample or additional sample.

In a further example, the method includes quantifying an amount and/or clonality of T-Cell and B-Cell receptors using digital PCR, targeted sequencing, or whole genome sequencing methods (WGS).

In either of the above examples the method may further include performing sodium bisulfite conversion of genomic DNA to differentiate and detect unmethylated versus methylated cytosines.

In a further example, the method includes performing either PCR amplification or massively parallel sequencing methods to reveal the methylation status of every cytosine in gene specific amplification or whole genome amplification.

In a further example, the method may include comparing the levels of gene specific DNA methylation with SARS-CoV-2 gene expression or amplification and the presence of HPV DNA or RNA, whereby if the levels of gene specific DNA methylation amplification in the sample is high in SARS-CoV-2 and HPV positive subjects, then the subject has an increased risk of accelerated pre-malignant progression, development of cancer, having cancer, or cancer progression.

In a further example of an above example, the method includes comparing the levels of whole genome DNA methylation with SARS-CoV-2 gene expression or amplification and the presence of HPV DNA or RNA, wherein if the levels of whole genome DNA methylation amplification in the sample is low in a SARS-CoV-2 and HPV positive subject, the subject has an increased risk of accelerated pre-malignant progression, development of cancer, having cancer, or cancer progression.

In a further example, the method may include comparing the levels of gene specific DNA methylation and host RNA expression levels with the presence of SARS-CoV-2 and HPV DNA or RNA, whereby if concordant levels of host DNA methylation and RNA expression levels in the sample are correlated (positively or negatively) with SARS-CoV-2 and HPV subjects, then the subject has an increased risk of accelerated pre-malignant progression, development of cancer, having cancer, or cancer progression.

In a further example, the RNA is one or more of mRNA, microRNA, or long-non-coding RNA.

In a further example the method includes comparing the amount and clonality of T-Cell receptors and B-Cell receptors in the samples with gene specific DNA methylation level, whereby, if gene specific DNA methylation is inversely correlated to T-Cell receptors and/or B-Cell receptors amount or clonality, then the subject has an increased risk of accelerated pre-malignant progression, development of cancer, having cancer, or cancer progression.

In any of the above examples or another example, comparing the presence, amount of SARS-CoV-2 nucleic acids, SARS-CoV-2 gene expression or amplification includes comparison to a predetermined level of HPV DNA or RNA. In a further example, the HPV has been previously quantified.

In one example, the genomic DNA is treated with affinity-based methods to differentiate and detect unmethylated versus methylated cytosines, such as Methylated DNA Immuno Precipitation (MEDIP) or Methylated DNA Binding proteins (MBD).

In one example, the genomic DNA is treated with enzymatic methods to differentiate and detect unmethylated versus methylated cytosines.

In another aspect, a method for detection of cancer risk mediated by SARS-CoV-2 and HPV includes quantifying, in a sample isolated from a subject, SARS-CoV-2 nucleic acids; analyzing a sample isolated from a subject for one or more host biomarkers associated with a risk of a cancer; and comparing an amount of SARS-CoV-2 nucleic acids in a sample to the presence of HPV DNA or RNA, wherein, if the value of SARS-CoV-2 nucleic acids in the sample is independently high or high relative to a quantification of the HPV DNA or RNA and the one or more biomarkers are detected in HPV positive subjects then the subject has an increased risk of premalignant progression associated with the cancer, having the cancer, or progression of the cancer.

In one example, the sample from which SARS-CoV-2 nucleic acids are quantified corresponds to a same tissue or biofluid from which the presence of HPV was quantified.

In the above or another example, the HPV has been previously quantified.

In any of the above or another example, comparing the amount of SARS-CoV-2 nucleic acids in the sample to the presence of HPV DNA or RNA may include comparing the levels of SARS-CoV-2 gene expression in a sample to the presence of HPV DNA or RNA and wherein the value comprises an expression value of the SARS-CoV-2 gene.

In any of the above or another example, the sample from which SARS-CoV-2 nucleic acids are quantified may corresponds to a same tissue or biofluid that is analyzed for at least one of the one or more biomarkers.

In any of the above or another example, the one or more biomarkers may include an expression value of one or more host genes in the sample that correlates (positively or negatively) in HPV and SARS-CoV-2 positive subjects.

In any of the above or another example, the one or more biomarkers may include a level of gene specific DNA methylation of one or more host genes and a host RNA expression level corresponding to the one or more genes that positively or negatively correlate with HPV infected subjects also infected with SARS-CoV-2 or having long COVID.

In any of the above or another example, the one or more biomarkers may include a differential promoter methylation of one or more host genes relative to corresponding samples of unaffected subjects.

In any of the above or another example, the one or more biomarkers may include an amount and/or clonality of T-Cell and/or B-Cell receptors in the sample, and wherein the method further includes analyzing the sample to quantifying an amount and/or clonality of T-cell and/or B-cell receptors in the sample.

In any of the above or another example, the one or more biomarkers may include a microbiota differential relative to corresponding samples of unaffected subjects, and wherein the method further comprises analyzing the sample for presence of a microbiota differential.

In any of the above or another example, the one or more biomarkers may include a predetermined low level of whole host genome DNA methylation in the sample relative to corresponding samples of unaffected subjects.

In any of the above or another example, the one or more biomarkers may include an inverse correlation between a gene specific DNA methylation with respect to one or more host genes and T-cell receptors and/or B-cell receptors amount or clonality.

In any of the above or another example, the sample comprises a first tissue or biofluid and a second tissue or biofluid.

In any of the above or another example, the method further includes isolating the sample from the subject.

In any of the above or another example, the sample comprises a cervical liquid cytology sample, saliva sample, urine sample, cervical smear, vaginal lavage fluid sample, anal smear, stool sample, tumor sample, tissue sample, or any combination thereof.

In any of the above or another example, the cancer is. oral cancer, tongue cancer. oropharyngeal cancer. anal cancer, penile cancer, vulvar cancer, or vaginal cancer.

In yet another aspect. a method for detection of cancer risk mediated by SARS-CoV-2 and HPV includes determining a subject has an increased risk of accelerated premalignant progression associated with a cancer, having the cancer, or progression of the cancer if the subject is HPV positive and a biospecimen sample isolated from the subject corresponding to a tissue or biofluid secreted or contacting tissue associated with the cancer if an amount of SARS-CoV-2 nucleic acids in the sample is independently high or high relative to an amount of HPV DNA or RNA in the sample or a previous sample a biospecimen sample isolated from the subject corresponding to a tissue or biofluid secreted or contacting tissue associated with the cancer.

### BRIEF DESCRIPTION OF THE DRAWINGS

FIG. 1 is a boxplot of Cycle Threshold (Ct) values for N and MS2 in 72 liquid cytology samples.

FIGS. 2A-2C are boxplots showing DNA promoter methylation in liquid cytology samples from women with No Intraepithelial Lesions or Malignancy (NILM) and Cervical Intraepithelial Neoplasm Grade 2 or Grade 3 lesions (CIN 2/3) pathology diagnosis for FKBP6 (FIG. 2A); ZNF516 (FIG. 2B); and INTS1 (FIG. 2C).

FIG. 2D is a Receiver Operator Characteristics (ROC) curve for panel of FKBP6, ZNF516, and INTS1 DNA promoter methylation in liquid cytology samples from women with NILM and CIN 2/3 pathology diagnosis.

FIG. 2E shows summary statistics for FKBP6, ZNF516, INTS1 promoter DNA methylation and N cycle threshold.

FIG. 3A depicts a linear relationship between the ZNF516 promoter DNA methylation in liquid cytology media and N gene Cycle Threshold (Ct) values in liquid cytology media.

FIG. 3B depicts a linear relationship between the INTS1 promoter DNA methylation in liquid cytology media and N gene Cycle Threshold (Ct) values in liquid cytology media.

FIG. 3C shows results of linear models (Pearson correlation, slope beta values and goodness-of-fit) for ZNF516 and INTS1 promoter DNA methylation with N cycle threshold values.

FIG. 4A is a graph depicting T-cell receptor (TCR) quantity and clonality in tumor infiltrating lymphocytes (TILs) oropharyngeal cancer patients positive to oncogenic Human Papilloma Virus (HPV+) compared with tumor tissue from HPV− oropharyngeal patients (p<0.05).

FIG. 4B is a graph depicting T-cell receptor quantity and clonality in tumor infiltrating lymphocytes (TILs) in head and neck cancer tissue samples (p<0.05) by short-term and long-term survival.

FIG. 4C is a graph depicting T-cell receptor quantity and clonality in tumor infiltrating lymphocytes (TILs) by anatomic site in head and neck cancer patients.

FIG. 4D is a graph depicting T-cell receptor quantity and clonality in tumor infiltrating lymphocytes (TILs) in paired lymphocyte and Head and neck cancer tissues.

FIG. 4E is a graph depicting T-cell receptor beta (TCR-b) chain clones' quantity in cervical intraepithelial Grade 1 and Grade 2 (CIN1/CIN2) lesions.

### DESCRIPTION

In various embodiments, the present description describes methods of detecting, which may include early detection, of cancer mediated by SARS-CoV-2 and HPV. The methods may include sample analysis directed to detection of such viral infections alone or together with one or more biomarkers. In various embodiments, the biomarkers may be selected from epigenomic factors such as one or more gene specific DNA methylation levels or whole genome DNA methylation level, host RNA expression levels. T-Cell receptor amount or clonality, B-Cell receptor amount or clonality, microbiome, and any combination thereof. One method includes analysis of SARS-CoV-2 nucleic acid and the one or more biomarkers from samples of the same tissue. biofluid, or both. These methods are useful for, among other things, assessing the effectiveness of treatment, monitoring relapse, and clinical staging of cancer. These methods are also useful for among other things to monitor the effectiveness of strategies and therapies used to modify lifestyle and contextual effects to prevent disease, foster wellness and enable health promotion.

In some embodiments. the methods disclosed herein may be employed for early cancer detection and follow-up of HPV related tumors in subjects with SARS-CoV-2 infection and Long COVID using saliva and other biofluids. In one example, disclosed methods may be utilized to addresses limitations of the current diagnostic methods by providing a non-invasive, cost-effective, and accurate method for early cancer detection and follow-up of HPV-related tumors in SARS-CoV-2 positive patients and patients with Long COVID. The present disclosure provides several advantages over conventional diagnostic methods, including non-invasiveness, reduced patient discomfort, and lower costs. Additionally, the invention is particularly useful for patients with SARS-CoV-2 infections and Long COVID, who may experience complications with invasive diagnostic procedures.

In various embodiments, the disclosed methods may be employed to detecting and monitoring viral and host biomarkers associated with cancer linked to SARS-CoV-2 and HPV, which can be used for early detection. monitoring of therapeutic effectiveness. and relapse monitoring of such cancers. In one embodiment, a method includes obtaining a biological sample from a subject, detecting the presence or absence of the viral and host biomarkers in the sample, and correlating the presence or absence of the biomarkers with the likelihood of developing cancer, the effectiveness of a therapeutic intervention, or the risk of cancer relapse.

In various embodiments, a method for detecting SARS-CoV-2 nucleic acids in a subject at risk of cancer linked to Human Papilloma Virus (HPV) infection includes (a) isolating RNA from a specimen; (b) using reverse transcription process to convert the RNA into cDNA; and (c) quantifying SARS-CoV-2 nucleic acids using Real-Time Polymerase Chain Reaction (RT-PCR). Loop Mediated Amplification (LAMP) or SARS-CoV-2 Whole Genome Sequencing (WGS); and (d) comparing the amount of SARS-CoV-2 nucleic acids in a sample to the presence of HPV DNA or RNA (predetermined level), wherein the HPV has been previously quantified, whereby if the value of SARS-CoV-2 nucleic acids in the sample is high in HPV positive subjects. then the subject has an increased risk of one or more of accelerated pre-malignant progression, developing cancer, having cancer, or cancer progression. In one example, the subject has an increased risk of accelerating pre-malignant progression of cancer. In one example, the subject has an increased risk of accelerated progression of cancer. In one example, the subject has an increased risk of developing cancer. In one example, the subject has an increased risk of having cancer. In one embodiment, the subject has an increased risk of cancer progression.

As used herein, the terms test subject. subject or patient are used interchangeably and refer to a human or another animal species, including primates, rodents (i.e., mice, rats, and hamsters), farm animals, sport animals and pets. In various embodiments, the subject is a human. In certain embodiments. the methods find use in experimental animals, in veterinary application, and/or in the development of animal models for disease.

As described in more detail below. in some embodiments. the methods may include utilization of saliva and/or other biofluids as potential surrogates for diagnostic immune profiling of tumors. For example, in various embodiments, the method comprises collecting saliva or other biofluid samples from a patient; analyzing the collected sample for the presence of specific biomarkers indicative of tumor immune profiles and HPV infection; correlating the detected biomarkers with the patient's clinical data, including SARS-CoV-2 infection status and Long COVID symptoms; and interpreting the data to determine the presence or absence of early-stage cancer or HPV-related tumors. In one example, if applicable. the method may include monitoring the progression or regression of the identified tumors over time through periodic follow-up testing.

In one embodiment, a method for detecting increased risk of having or developing cancer in a subject includes (a) isolating a RNA sample from a subject at risk of cancer linked to HPV infection; (b) convening the RNA into cDNA; (c) quantifying the expression of SARS-CoV-2 S, N, ORF and/or E genes using Real-Time Polymerase Chain Reaction (RT-PCR), Loop Mediated Amplification (LAMP) or SARS-CoV-2 Whole Genome Sequencing (WGS); and (d) comparing the levels of SARS-CoV-2 gene expression in a sample to the presence of HPV DNA or RNA (predetermined level), wherein the HPV has been previously quantified, whereby if the expression value of SARS-CoV-2 genes in the sample is high in HPV positive subjects, then the subject has an increased risk of one or more of accelerated pre-malignant progression, developing cancer, having cancer, or cancer progression. In one example, the subject has an increased risk of accelerating pre-malignant progression of cancer. In one example, the subject has an increased risk of accelerated progression of cancer. In one example, the subject has an increased risk of developing cancer. In one example, the subject has an increased risk of having cancer. In one embodiment, the subject has an increased risk of cancer progression.

In various embodiments, the methods disclosed herein include identification of a cancerous cell or a pre-cancerous cell in a background of SARS-CoV-2 infection having differential DNA methylation values when compared to a cancerous cell or a pre-cancerous cell that is not in a background of SARS-CoV-2 infection, as determined by the presence of SARS-CoV-2 nucleic acids.

In various embodiments, the methods disclosed herein include identification of a DNA methylation signature. The DNA methylation signature may be derived, for example, from healthy cells or a healthy tissue is altered in a background of SARS-CoV-2 infection, as determined by the presence of SARS-CoV-2 nucleic acids.

In various embodiments, a method for screening for increased risk of one or more of accelerating pre-malignant progression, accelerating progression, developing, or having cancer in a subject with SARS-CoV-2 infection, as determined by the presence of SARS-CoV-2 nucleic acids includes (a) isolating a DNA sample from a subject; (b) measuring a DNA methylation signature in a sample; (c) measuring the presence of SARS-CoV-2 nucleic acids; and (d) comparing the values of DNA methylation across the epigenome in a sample with a background of SARS-CoV-2 infection to a the DNA methylation signature in samples taken from a healthy subject or a pool of subjects without SARS-CoV-2 infection, whereby if the value of DNA methylation in the sample is different in the subject with SARS-CoV-2 infection, than the DNA methylation value in subject(s) without SARS-CoV-2 infection, then the subject has an increased risk of one or more of accelerated pre-malignant progression, developing cancer, having cancer, or cancer progression. In one example, the subject has an increased risk of accelerating pre-malignant progression of cancer. In one example, the subject has an increased risk of accelerated progression of cancer. In one example, the subject has an increased risk of developing cancer. In one example, the subject has an increased risk of having cancer. In one embodiment, the subject has an increased risk of cancer progression.

In various embodiments, a method for assessing the risk of having cancer in a subject, comprising or consisting of: (a) isolating a DNA sample from a subject; (b) measuring a DNA methylation signature in a sample; (c) measuring the presence of SARS-CoV-2 nucleic acids; and (d) comparing the values of DNA methylation across the epigenome in a sample with a background of SARS-CoV-2 infection to a the DNA methylation signature in samples taken from a healthy subject or a pool of subjects without SARS-CoV-2 infection, whereby if the value of DNA methylation in the sample is different in the subject with SARS-CoV-2 infection, than the DNA methylation value in subject(s) without SARS-CoV-2 infection, then the subject has an increased risk of one or more of accelerated pre-malignant progression, developing cancer, having cancer, or cancer progression. In one example, the subject has an increased risk of accelerating pre-malignant progression of cancer. In one example, the subject has an increased risk of accelerated progression of cancer. In one example, the subject has an increased risk of developing cancer. In one example, the subject has an increased risk of having cancer. In one embodiment, the subject has an increased risk of cancer progression.

In one embodiment, the present disclosure provides a method for screening for increased risk of one or more of accelerating pre-malignant progression. accelerating progression, developing, or having cancer in a subject with SARS-CoV-2 and HPV infection, as determined by the presence of SARS-CoV-2 and HPV nucleic acids, comprising or consisting of: (a) isolating a DNA sample from a subject; (b) measuring a DNA methylation signature in a sample; (c) measuring the presence of SARS-CoV-2 nucleic acids; (d) measuring the presence of HPV nucleic acids; and (d) comparing the values of DNA methylation across the epigenome in a sample with a background of SARS-CoV-2 and HPV infection to the DNA methylation signature in samples taken from a healthy subject or a pool of subjects without SARS-CoV-2 and HPV infection, whereby if the value of DNA methylation in the sample is different in the subject with SARS-CoV-2 and HPV infection, than the DNA methylation value in subject(s) without SARS-CoV-2 and HPV infection, then the subject has an increased risk of one or more of accelerated pre-malignant progression, developing cancer, having cancer, or cancer progression. In one example, the subject has an increased risk of accelerating pre-malignant progression of cancer. In one example, the subject has an increased risk of accelerated progression of cancer. In one example, the subject has an increased risk of developing cancer. In one example, the subject has an increased risk of having cancer. In one embodiment, the subject has an increased risk of cancer progression.

In one embodiment, the present disclosure provides a method for assessing a cancer risk in a subject includes (a) isolating a DNA sample from a subject; (b) measuring a DNA methylation signature in a sample; (c) measuring the presence of SARS-CoV-2 nucleic acids; (d) measuring the presence of HPV nucleic acids; and (d) comparing the values of DNA methylation across the epigenome in a sample with a background of SARS-CoV-2 and HPV infection to a the DNA methylation signature in samples taken from a healthy subject or a pool of subjects without SARS-CoV-2 and HPV infection, whereby if the value of DNA methylation in the sample is different in the subject with SARS-CoV-2 and HPV infection, than the DNA methylation value in subject(s) without SARS-CoV-2 and HPV infection, then the subject has an increased risk of one or more of accelerated pre-malignant progression, developing cancer, having cancer, or cancer progression. In one example, the subject has an increased risk of accelerating pre-malignant progression of cancer. In one example, the subject has an increased risk of accelerated progression of cancer. In one example, the subject has an increased risk of developing cancer. In one example, the subject has an increased risk of having cancer. In one embodiment, the subject has an increased risk of cancer progression.

In an example of the above embodiments, the DNA methylation signature comprises promoter methylation of one or more biomarkers of premalignant progression from intraepithelial lesions to intraepithelial neoplasia, cervical dysplasia, cancer progression, or disease. In one example, the DNA methylation signature comprises promoter methylation of the human genes ZNF516, FKBP6 or INTS1, or their homologs, orthologs, or analogs. In an above or another example, the DNA methylation signature is measured using quantitative Real Time Methylation Specific PCR (qMSP).

Detecting or quantifying SARS-CoV-2 according to the methods described herein may be performed according to any suitable methodology. For example, presence may be identified via an antigen test or RT-PCR may be used to amplify SARS-CoV-2 RNA in the sample. Additional examples for quantifying SARS-CoV-2 include Loop Mediated Amplification (LAMP) or SARS-CoV-2 Whole Genome Sequencing (WGS). In one example, a TaqPath COVID-19 Combo kit designed to coamplify sections of three SARS-CoV-2 viral genes: Nucleocapsid (N), Open Reading Frame lab (ORFlab), and Spike (S) may be used. In an example of the above embodiments, quantifying SARS-CoV-2 nucleic acids using Real-Time Polymerase Chain Reaction (RT-PCR) or measuring the presence of SARS-CoV-2 nucleic acids comprises determining a threshold cycle (Ct) value. In various embodiments, the same sample or another sample from the same cell or tissue that is assayed to detect biomarkers is also used with respect to detection or quantification of SARS-CoV-2.

In various embodiments, the methods described herein include identification of an HPV status. Identification may include identification of a currently known status or determination of such a status, e.g., by optical. visual, clinical, pathological, or other suitable means. Status may include, for example, one or more of an HPV positive or negative status, a quantification of HPV with respect to a tissue, organ, system. or body fluid, an identification or detection of an HPV type, or any combination thereof. HPV may be detected by presence of HPV DNA in the cervix. Some examples of HPV detection include the Hybrid Capture II (HCII) assay, amplification of viral DNA using PCR techniques. and detection of mRNA. In one embodiment, the methods may include quantifying circulating HPV DNA in urine. HPV typing may include determination of an HPV type of an HPV positive subject. e.g., via sequencing and/or RT-PCR. For example, HPV types 16, 18, 31, 33, 35, 39, 45, 51, 52, 56, 58, 59, 66. and 68 may be correlated with higher risk of cancer or premalignant development than other types.

In various embodiments, the same sample or another sample from the same cell or tissue that is assayed to detect biomarkers is also used with respect to detection or quantification of one or both, HPV and/or SARS-CoV-2.

In some embodiments, the subject is determined to have an increased risk of accelerated premalignant progression associated with a cancer, having the cancer, or progression of the cancer when the subject is HPV+ and currently has COVID or Long COVID if an amount of SARS-CoV-2 nucleic acids in current or previous sample is high or is associated with a high characterization relative to the amount of HPV DNA or RNA.

In one embodiment, the present disclosure provides a method for early cancer detection and follow-up of HPV-related tumors in SARS-CoV-2 positive patients and patients with Long COVID using saliva and other biofluids. The method may include one or more of sample collection, sample analysis, data correlation, data interpretation, follow-testing, or combination thereof.

In one example, sample collection includes collection of saliva or other biofluid samples, such as blood, urine, or sputum, from a subject using non-invasive techniques. The samples can be collected by the subject themselves or by a healthcare professional.

In one example, sample analysis may include analysis of the collected sample for the presence of specific biomarkers indicative of tumor immune profiles and HPV infection. Such biomarkers may include, but are not limited to, tumor-associated antigens, cytokines, chemokines, immune cell populations, or other biomarkers, such as those described elsewhere herein. The analysis may be performed using known techniques such as enzyme-linked immunosorbent assay (ELISA), polymerase chain reaction (PCR), mass spectrometry, or other suitable methods.

In one example, data correlation includes correlating the detected biomarkers with the subject's clinical data. The clinical data may include SARS-CoV-2 infection status and/or Long COVID symptoms. This correlation may be used to enable identification of patterns indicative of early-stage cancer or HPV-related tumors in the context of SARS-CoV-2 infection and Long COVID.

In one example, data interpretation includes determining presence or absence of early-stage cancer or HPV-related tumors in the data obtained from the biomarker analysis and correlation with clinical data. In a further example, this interpretation incorporates machine learning algorithms, statistical models, or other suitable methods to accurately identify the presence or risk of cancer or HPV-related tumors in the patient.

In one example, when applicable, follow-up testing may include monitoring the progression or regression of the identified tumors over time through periodic follow-up testing, which in one configuration uses the same or similar methodology as described above. This follow-up testing enables healthcare professionals to assess the effectiveness of treatments and to adjust treatment plans accordingly.

The present disclosure provides several advantages over conventional diagnostic methods. The use of saliva and other biofluids, for example, as potential surrogates for diagnostic immune profiling of tumors is non-invasive diagnostic procedures reduces patient discomfort and lowers the risk of complications associated with invasive diagnostic procedures. Additionally, the methods may be implement more cost-effectively compared to traditional imaging techniques and biopsies. Such a method may be particularly useful for subjects with SARS-CoV-2 infections and Long COVID, who may experience complications with invasive diagnostic procedures. By using saliva and other biofluids, this method allows for early cancer detection and follow-up of HPV-related tumors in a safer and more convenient manner for these patients.

In one embodiment, a method for detecting and monitoring viral and host biomarkers associated with cancer linked to SARS-CoV-2 and HPV includes (a) obtaining a biological sample from a subject; (b) detecting the presence or absence of viral and host biomarkers in the biological sample; and (c) correlating the presence or absence of the viral and host biomarkers with the likelihood of developing cancer, the effectiveness of a therapeutic intervention, or the risk of cancer relapse.

In one example, the biological sample is selected from the group comprising or consisting of blood, saliva, urine, and tissue biopsy. In the above or another example, the viral biomarkers comprise viral nucleic acids, viral proteins, or viral antigens associated with SARS-CoV-2 or HPV. In any of the above or another example, the host biomarkers comprise proteins, nucleic acids, or other molecules indicative of a host response to viral infection, cancer development, or cancer progression. In any of the above or another example, the method further includes using of a diagnostic kit for the detection and monitoring of viral and host biomarkers, wherein the diagnostic kit comprises reagents and materials necessary for the analysis of the biological sample and a device or apparatus for the detection of the biomarkers. In any of the above or another example, the method further includes integrating the method with other diagnostic techniques, such as imaging or histopathology, to provide a comprehensive evaluation of the subject's cancer status, therapeutic response, and risk of relapse. In one any of the above or another example, the cancers are linked to SARS-CoV-2 include lung cancer or other respiratory tract cancers. In one any of the above or another example, the cancers are linked to HPV include cervical, anal, or oropharyngeal cancer. In one any of the above or another example, the method is employed for the early detection of cancer linked to SARS-CoV-2 or HPV by detecting the presence or absence of viral and host biomarkers in a biological sample obtained from a subject. In one any of the above or another example, the method is employed for the monitoring of therapeutic effectiveness in cancer linked to SARS-CoV-2 or HPV by detecting changes in the levels of viral and host biomarkers over time and correlating these changes with the subject's response to therapy. In one any of the above or another example, the method is employed for the relapse monitoring of cancer linked to SARS-CoV-2 or HPV by detecting changes in the levels of viral and host biomarkers over time and correlating these changes with the risk of cancer relapse.

In another embodiment, the present disclosure comprises a diagnostic kit include the materials, reagents, and biological materials for performing any of the methods disclosed herein.

In one embodiment, a diagnostic kit for detecting and monitoring viral and host biomarkers associated with cancer linked to SARS-CoV-2 and HPV includes (a) reagents and materials necessary for the analysis of a biological sample, including but not limited to, primers, probes, antibodies, enzymes, buffers, and other consumables; (b) a device or apparatus for the detection of viral and host biomarkers, such as a PCR machine, an enzyme-linked immunosorbent assay (ELISA) plate reader, or a mass spectrometer; and, optionally, (c) instructions for use of the diagnostic kit.

In certain embodiments. the methods described herein comprise detecting cancer or any increased risk of having or developing cancer. The cancer can be any neoplastic disease, including carcinoma and solid tumors. The term “cancer” is also meant to include metastatic disease, metastases, and metastatic lesions, which are groups of cells that have migrated to a site distant relative to the primary tumor. In one embodiment, the cancer is a solid tumor. In one embodiment, the cancer is characterized by comprising a metastatic cancer cell population.

In various embodiments, the cancer is cervical cancer. In one embodiment, the cancer is oropharyngeal cancer. In some embodiments, the cancer is anal cancer. In one embodiment, the cancer is penile cancer. In one embodiment. the cancer is vaginal cancer. In an embodiment. the cancer is vulvar cancer.

In various embodiments, cancers may include squamous cell carcinomas (SCC), such as one or more of squamous cell originating forms of hypopharyngeal cancer, laryngeal cancer, lip cancer, oral cancer, nasopharyngeal cancer, oropharyngeal cancer, paranasal sinus cancer, cancer of the nasal cavity, salivary gland cancer, lung cancer, penile cancer, vaginal cancer, rectal cancer, colon cancer, or pancreatic cancer, or any combination thereof.

In various embodiments, cancers may include head and neck cancers, such as one or more of mouth cancer, throat cancer, oral cancer, laryngeal cancer, lip cancer, nasal cancer, paranasal cancer, salivary gland cancer, nasopharyngeal cancer, hypopharyngeal cancer, other head and neck cancers, or any combination thereof.

In various embodiments, cancers may include HPV linked cancers. such as one or more of gastric cancer, liver cancer, cervical cancer, vulva cancer, penile cancer, vaginal cancer, anal cancer, lung cancer, head and neck cancers, such as oropharyngeal cancer, oral cancer, throat cancer, tongue cancer, or tonsil cancer, or any combination thereof.

In various embodiments, cancers include SCC linked to HPV. such as one or more of gastric cancer, cervical cancer, vulva cancer, vaginal cancer, penile cancer, head and neck squamous cell carcinomas (HNSCC) such as oropharyngeal cancer, or any combination thereof.

In one embodiment, cancers include HPV linked cancers selected from cervical cancer, oral cancer, gastric cancer, liver cancer, and any combination thereof.

In one embodiment, cancers include head and neck cancers.

In one embodiment, cancers include HPV linked head and neck cancers selected from oropharyngeal cancer, oral cancer, and any combination thereof. In one example, oropharyngeal cancer or oral cancer include throat cancer, tongue cancer, or tonsil cancer, or any combination thereof.

In some embodiments, the methods described herein include identification of a current cancer status with respect to one or more cancers. Identification, which may include detecting, may include identification of a currently known status or determination of such a status, e.g., by optical, visual. clinical. pathological, or other suitable means. In one example, the identification may be used for comparative analyses to standard samples to detect correlations between viral presence or levels/loads, identify biomarkers for use in the disclosed methods, measure or detect risks with respect to conditions for future screening analysis of samples from subjects in which a current cancer status is not known. In one example, the identification comprises a performing or recommending a status determination procedure. which may include testing, based on a detected risk resulting from the method described herein.

The methods of the of the present disclosure may comprise using samples comprising or consisting of biospecimens collected from one or more of patients or subject animals, a cell culture, or a tissue culture. Biospecimens may include, for example, tissues, biofluids, or both. In various embodiments, one or more samples may be selected from a cervical liquid cytology sample, saliva sample, urine sample, cervical smear, vaginal lavage fluid sample, anal smear, stool sample, tumor sample, tissue sample, or any combination thereof. According to one embodiment, the methods include selecting one or more of samples comprising a saliva sample for head and neck cancer; cervical epithelium scrapes. liquid cytology sample transport media (such as PreservCyt, ThinPrep and SurePath), and/or vaginal swabs (dry or in sample transport media) for cervical cancer; anal scrapes and/or swabs (dry or in sample transport media) for anal cancer, penile scrapes and/or swabs (dry or in sample transport media) for penile cancer; or blood and/or urine for head and neck, cervical, anal and penile cancer. In various embodiments, tissue samples may include tissues associated with the cancer for which the risk is to be detected. For example, tissue samples may include cervical tissue for cervical cancer, oropharynx tissue for oropharyngeal cancer, throat tissue for throat cancer, or oral cavity tissue for oral cancer.

The samples may be analyzed for one or more of presence, quantity, location, or distribution of nucleic acids (viral or host), transcription, gene expression, genetic mutations, epigenomic factors related to one or more of HPV, SARS CoV-2, or biomarkers associated with cancer, e.g., pre-malignant progression or acceleration thereof, which may include risk of developing cancer, having cancer, which may further include progression of cancer. Biomarkers may include, for example, epigenomic factors such as one or more gene specific DNA methylation levels or whole genome DNA methylation levels, host RNA expression levels, T-Cell receptor amount or clonality, B-Cell receptor amount or clonality, microbiome, or any combination thereof. The analysis may be performed by any methodology disclosed herein, currently known, or known in the future.

In various embodiments, sample analysis results with respect to a subject may be compared with standard cell or tissue analyses. In this or another embodiment. the method includes identifying biomarkers comprising comparing analyses of standard cell or tissue samples with samples from subjects known to have a condition associated with cancer, e.g., pre-malignant progression or acceleration thereof, which may include risk of developing cancer, having cancer, which may further include progression of cancer. In one embodiment, the comparative analyses may be used to identify biomarkers, establish baseline standards for comparative analyses, identify relative correlation levels between the biomarkers and conditions for determining associated risks in samples, develop risk scores with respect to the conditions, or any combination thereof.

In various embodiments, a standard cell or tissue is a non-cancerous cell or tissue. In one embodiment, a standard cell or tissue is a non-neoplastic cell or tissue. In some embodiments, a standard cell or tissue is a non-cancerous differentiated or non-differentiated cell or tissue. In one embodiment, the standard is derived from non-cancerous differentiated or non-differentiated cells or tissues. In various embodiments, the sample and standard are derived from a common cell or tissue but from different sources wherein the standard is derived from a non-cancerous tissue. In one embodiment, the sample and standard are derived from a common tissue but from different sources wherein the standard is derived from a non-cancerous tissue and the sample is from a subject having cancer or suspected of being afflicted with cancer. In various embodiments, the sample and standard are derived from a common tissue and a common source wherein the standard is derived from a non-cancerous cell and the sample is derived from cells suspected of being cancerous cells.

In various embodiments, the sample is collected after a surgical treatment. In one embodiment, the sample is collected after a radiation therapy. In one embodiment, the sample is collected after a chemotherapy treatment. In some embodiments, the sample is collected before a surgical treatment. In one embodiment, the sample is collected before a radiation therapy. In an embodiment, the sample is collected before a chemotherapy treatment. In some embodiments, a sample is collected before and after a surgical treatment. In one embodiment, a sample is collected before and after a radiation therapy. In various embodiments, a sample is collected before and after a chemotherapy treatment.

In various embodiments, biomarkers utilized according to the present methods may be associated with premalignant lesions, cancer, cancer development, tumor aggressiveness, invasiveness, malignant transformation, early detection of primary or relapsing carcinomas, or any combination thereof with respect to one or more cancers.

In various embodiments the subject has epigenetic changes as a result of exposure to stressful biopsychosocial causal factors, such as, but not limited to, diseases associated to elevated allostatic load, which is linked to the social environment of poor inner-city neighborhoods, remote poor rural areas or marginalized urban sectors that lack social cohesion and have high rates of criminality, abandoned buildings, drug addiction and poverty.

In various embodiments, an epigenetic change in a subject indicates that the subject has an increased risk of being afflicted with cancer. In one embodiment, an epigenetic change in a subject indicates that the subject has an increased risk of developing cancer.

According to various embodiments, biomarkers include differentially methylated regions (DMRs). In an example of the above embodiments, the DNA methylation signature comprises DMRs corresponding to promoter methylation of one or more genes that serve as a biomarker of premalignant progression from intraepithelial lesions to intraepithelial neoplasia, cervical dysplasia, cancer progression, or disease. Detection or quantification of DNA methylation may be determined by any suitable methodology. In one example, genomic DNA is treated with affinity-based methods to differentiate and detect unmethylated versus methylated cytosines, such as Methylated DNA Immuno Precipitation (MEDIP) or Methylated DNA Binding proteins (MBD). In a further or another example, genomic DNA is treated with enzymatic methods to differentiate and detect unmethylated versus methylated cytosines. In a further or another example, genomic DNA is subjected to sodium bi-sulfite conversion to differentiate and detect unmethylated versus methylated cytosines. In a further or another example, PCR amplification or massively parallel sequencing methods are used to reveal the methylation status of every cytosine in gene specific amplification or whole genome amplification. In one example, DNA bisulfite conversion (using EpiTect Fast LyseAll Bisulfite Kit (QIAGEN)) is performed and followed by DNA methylation profiling (Methylation Specific PCR (qMSP) of bisulfite-modified genomic DNA optimized for QuantStudio™ 6 Flex (Thermo Fisher Scientific)). PCR may utilize primers and probes designed to specifically amplify the promoters of specific target genes of interest may be used.

In various embodiments of the methods described herein a DNA methylation signature biomarker may comprise promoter methylation of specific genes or their homologs, orthologs, or analogs. In one example, the cancer comprises cervical cancer and the DNA methylation signature biomarker comprise promoter methylation of ZNF516, FKBP6 and/or INTS1, In one example, a DNA methylation signal may correspond to a differential methylation of biomarkers comprising NID2, HOXA9, or both. wherein promoter hypermethylation is associated with increased risks associated with oral squamous cell carcinoma. According to one embodiment, promoter hypermethylation of NID2, HOXA9, or both is measured from saliva, oral cavity tissues, or both. In one embodiment biomarkers for head and neck squamous cell carcinomas include one or more human genes selected from DAPK1, CDH1, PAX1, CALCA, TIMP3, and any combination thereof. In an above or another example, the DNA methylation signature is measured using quantitative Real Time Methylation Specific PCR (qMSP). In one embodiment, quantification of genome wide DNA methylation may be used as a biomarker.

Identification of DMRs for use as biomarkers may be performed by any suitable methodology. In one example, a genome wide differential methylation analysis and/or a gene ontology analysis may be performed to identify candidate genes to test for prognostic value and association with clinicopathological features. For instance, samples from known healthy subjects and samples from subjects known to have conditions for which the biomarkers are to be used to detect risk in connection with SARS-CoV-2 and HPV criteria may be hybridized to a genome-wide tiling array to identify statistically significant DMRs in a discovery cohort. Quantitative Methylation Specific PCR (qMSP) may be used with primers and probes designed to quantify promoter methylation of the biomarker genes. Downstream analyses may then be used to label differences in promoter DNA methylation patterns for biomarker identification. As described above. the subject samples, including those of standard samples, may be collected or derived from a common cell or tissue. In some embodiments, the identify biomarkers are used in the present methods with samples collected or derived from the same or similar common cell or tissue. In one example, the common cell or tissue corresponds to a cell or tissue affected by the potential cancer for which a risk is associated.

In one embodiment, biomarker identification to identify differentially methylated genes to distinguish between conditions, e.g., presence of cancer, and normal tissue may account for populations with different risk factors. For example, different biomarkers may be identified, e.g., as described above, for populations with similar risk factors and used in the methods described herein to determine risk in populations having similar risk factors. In a further embodiment, a heterogeneous risk factor approach is used whereby, in a first stage, initial samples are collected from a population with high risk of the condition due to a set of similar risk factors. In the second stage, promoter methylation status of the best performing hypermethylated genes identified in the first stage are used to screen DNA isolated from a separate cohort of tumor samples from another population with a well-characterized histopathology. Markers that perform well in a population with a heterogeneous risk profile in a clinical setting in the first phase have a higher probability of performing well in a well-characterized set of confirmed cases and controls in the second phase.

In various embodiments, the methods include detection of immune-oncology biomarkers. For example, immune-oncology biomarkers may comprise T-Cell receptor quantity and/or clonality, B-Cell receptor quantity and/or clonality, or any combination thereof. Quantification of an amount and/or clonality of T-Cell and/or B-Cell receptors may be accomplished using any suitable methodology. For example, quantification of an amount and/or clonality of T-Cell and/or B-Cell receptors may be performed using digital PCR. targeted sequencing, or whole genome sequencing methods (WGS). For instance. T-cell clones may be tracked by determining T-cell receptor (TCR) rearrangements composed of variable (V)-diversity (D)-joining (J) region genes, which generate the antigen-specific complementarity determining region 3 (CDR3).

As described in more detail below, the methods may include use of one or more biofluids, such as saliva and one or more additional biofluids, as surrogates for diagnostic immune-profiling of tumors, for early cancer detection and follow-up of HPV related tumors in SARS-COV-2 positive patients or patients with Long COVID. who are also HPV positive.

In various embodiments, the methods include detection of microbiome biomarkers. Microbiome biomarkers may be identified via differential microbiota analysis of samples of known healthy subjects may be compared to samples of subjects known to have conditions for which the biomarkers are to be used in the disclosed methods to detect differentially enriched or reduced species relative to normal microbiota. In one example, Resphera Insight an ultrahigh resolution taxonomic assignment algorithm for 16S rRNA sequences may be used to for reliable taxonomic identification to the species level.

The biomarkers may be selected as determinants the relevant condition or condition progression corresponding to the risk intended to be detected. Microbiome biomarkers may be cell, tissue, or location specific such that the differential microbiota representing the biomarker may be present at one or more cells, tissues, or locations while absent with respect to one or more other cells, tissues, or locations.

The cell, tissue, or location and type of sample used according to the methods described herein may vary. The biospecimen samples may include one or both of tissue or biofluid, such as those described herein. The cell, tissue, or location may correspond to the cell, tissue, or location for which the biomarker was identified. In one example, the location is chosen as a biofluid, or tissue physiologically associated with the cancer locale. For example, biofluids in proximity or secreted by affected tissues, associated organs, or adjacent thereto.

In one embodiment, microbiota in samples collected from different locations of the subject may be compared for differential presence as a biomarker. For example, a biomarker may include differential presence of Actinobacteria in oral cavity tissue samples, oropharyngeal tissue samples, or both compared to nasopharyngeal and larynx tissue samples, wherein risks associated with of oral or oropharyngeal cancer are elevated when Actinobacteria is enriched in oral cavity tissue samples, oropharyngeal tissue samples, or both when compared to nasopharyngeal and larynx tissue samples.

As introduced above, microbiome analysis may be performed on biofluids such as fecal/stool, urine, saliva, vaginal fluid, blood, urine, or any combination thereof. In various embodiments, samples may include oral tissue or saliva microbiota sample for oral cancer, fecal microbiota sample for colon cancer or rectal cancer, intestinal microbiota sample for pancreatic cancer, saliva microbiota sample for head and neck cancer; cervical epithelium scrapes, liquid cytology sample transport media (such as PreservCyt, ThinPrep and SurePath), and/or vaginal swabs (dry or in sample transport media) for cervical cancer; anal scrapes and/or swabs (dry or in sample transport media) for anal cancer; penile scrapes and/or swabs (dry or in sample transport media) for penile cancer; and blood and/or urine microbiota sample for head and neck, cervical, anal and penile cancer.

In various embodiments, microbiome biomarkers for head and neck squamous cell carcinomas include one or more of depleted Actinomyces in tumor tissue, enriched Lactobacillus in saliva and/or tissues, Fusobacterium nucleatum, an oral cavity flora commensal bacterium linked to colon cancer, enriched in saliva, Streptococcus_salivarius: Streptococcus_vestibularis, Fusobacterium nucleatum, Prevotella oris, Rothia_mucilaginosa, Lactobacillus_gasseri:Lactobacillus_johnsonii, Lactobacillus fermentum, Lactobacillus rhamnosus, Lactobacillus spp, Parvimonas micra, Streptococcus mutans, and/or Fusobacterium nucleatum enriched in saliva, enriched Parvimonas in tissues, depletion of Fusobacterium periodonticum, Leptotrichia trevisanii, Leptotrichia hofstadii, and Leptotrichia buccalis, or any combination thereof. In one example, a microbiome biomarker includes Lactobacillus gasseri/johnsonii and/or Lactobacillus vaginalis in saliva with respect to oropharyngeal cancer.

In some embodiments, microbiome biomarkers comprise a lack of microbiome diversity relative to normal microbiomes.

In some embodiments, biomarkers may include additional or alternative biomarkers such as genetic mutations, promoter methylation, or other inactivation or suppression of tumor suppressor genes or pathways, e.g., p53 or retinoblastoma. In one embodiment. biomarkers may include one or both of detection or measurement of oncoprotein expression, e.g., E6 or E7 with respect to risks associated with head and neck squamous cell carcinomas in samples. In some embodiments. biomarkers comprise tumor-associated antigens, cytokines, chemokines, or the like.

As identified above and elsewhere herein, the methods may include collection, isolation. and/or analysis of a sample. In various embodiments, the sample comprises or consists of one or more tissues. In one example, the tissue comprises or consists of epithelial tissue. In one example, the tissue comprises or consists of squamous cells. In one example, the tissue comprises or consists of a body membrane. In one example, the tissue comprises or consists of connective tissue. In one example, the tissue comprises or consists of mucous membranes. In one example, the tissue comprises or consists of synovial membranes. In one example, the tissue comprises or consists of serous membranes. In one example, the tissue comprises or consists of muscle tissue. In one example, the tissue comprises or consists of tumor tissue (cell mass). In one example, the tissue comprises or consists of one or more tissues selected from the above tissues or other tissues disclosed herein.

In various embodiments. the sample comprises or consists of one or more biofluids. In one example, the biofluid comprises or consists of urine. In one example, the biofluid comprises or consists of blood. In one example, the biofluid comprises or consists of saliva. In one example, the biofluid comprises or consists of vaginal fluid. In one example, the biofluid comprises or consists of cervical mucus. In one example, the biofluid comprises or consists of sinus mucus. In one example, the biofluid comprises or consists of stomach fluid. In one example, the biofluid comprises or consists of intestinal mucus. In one example, the biofluid comprises or consists of fecal matter. In one example, the biofluid comprises or consists of sweat.

In various embodiments, the sample may comprise the relevant tissues and/or biofluids above from one or more locations of the subject's body. In one example, the sample comprises or consists of a cervical liquid cytology sample. In one example, the sample comprises or consists of a cervical smear. In one example, the sample comprises or consists of a vaginal lavage fluid sample. In one example, the sample comprises or consists of an anal smear. In one example, the sample comprises or consists of a stool sample. In one example, the sample comprises or consists of a tumor sample. In one example, the sample comprises or consists of a tissue sample taken from a body location or organ corresponding to a cancer for which risk is to be detected. In one example, the sample comprises or consists of tissue sample taken from an adjacent body location corresponding to a cancer for which risk is to be detected. In one example, the sample comprises or consists of a biofluid sample taken from a body location or organ corresponding to a cancer for which risk is to be detected. In one example, the sample comprises or consists of a biofluid sample secreted from a body location or organ corresponding to a cancer for which risk is to be detected. In one example, the sample comprises or consists of vaginal swab. In one example, the sample comprises or consists of penile scrape. In one example, the sample comprises or consists of cervical epithelium scrape. In one example, the sample comprises or consists of penile swab. In one example, the sample comprises or consists of cervical tissue. In one example, the sample comprises or consists of oropharynx tissue. In one example, the sample comprises or consists of oral cavity tissue. In one example, the sample comprises or consists of throat tissue. In one example, the sample comprises or consists of tongue tissue. In one example, the sample comprises or consists of lung tissue. In one example, the sample comprises or consists of sputum. In one example, the sample comprises or consists of lymph tissue.

As identified above and elsewhere herein, the methods may analyze samples for one or more biomarkers in one or more of the biomarker categories including gene specific DNA methylation levels, whole genome DNA methylation levels, host RNA expression levels, T-Cell receptor amount or clonality, B-Cell receptor amount or clonality, differential microbiome analysis with respect to enrichment, depletion. diversity, and presence relative to other bacteria. For example, a method may include analysis of a sample for gene specific methylation level and analysis of the same or different sample for T-Cell receptor amount and/or clonality. In one such example, the method includes analysis of a cervical swab or smear for gene specific promoter methylation of one or more biomarker genes and T-Cell receptor amount and/or clonality. In a further example, the sample is also analyzed for presence, quantity, and/or expression of SARS-CoV-2 and/or HPV DNA or RNA. In a further example, the microbiome of the sample is analyzed in addition to or alternatively to T-Cell receptor amount and/or clonality. In a further example, a saliva sample is analyzed for differential microbiota in addition to or alternatively to differential microbiota analysis. gene specific promoter methylation analysis of one or more biomarker genes, and/or T-Cell receptor amount and/or clonality of the cervical swap or smear sample. One or both of the samples may be analyzed for presence, quantity, and/or expression of SARS-CoV-2 and/or HPV DNA or RNA. In some embodiments, presence, quantification, and/or expression of HPV may already be known, e.g., from a previous pap smear.

As identified above and elsewhere herein, the methods may include detection of a cancer risk via analysis of the sample. e.g., for presence, quantity, or other characteristic with respect to one or more biomarkers, which may also include analysis for SARS-CoV-2 presence, quantification, and/or expression and/or HPV presence, quantification, and/or expression. In various embodiments, the cancer risk detected according to the methods described herein may include head and neck cancers, squamous cell carcinomas (SCC). HPV mediated cancers, or any combination thereof. In one example, the cancer comprises or consists of head and neck cancers. In one example, the cancer comprises or consists of throat cancer. In one example, the cancer comprises or consists of oral cancer. In one example, the cancer comprises or consists of laryngeal cancer. In one example, the cancer comprises or consists of gastric cancer. In one example, the cancer comprises or consists of liver cancer. In one example, the cancer comprises or consists of cervical cancer. In one example, the cancer comprises or consists of tongue cancer. In one example, the cancer comprises or consists of vulva cancer. In one example, the cancer comprises or consists of penile cancer. In one example, the cancer comprises or consists of vaginal cancer. In one example, the cancer comprises or consists of anal cancer. In one example, the cancer comprises or consists of lung cancer. In one example, the cancer comprises or consists of oropharyngeal cancer.

In various embodiments, the methods may be used as a genomic/epigenomic cancer screening and/or detecting tool for early detection of every cancer site/type linked to HPV infection. In one embodiment, the methods may be used as an epigenomic cancer screening and/or detecting tool of cancer recurrence after treatment of a primary tumor. as a biomarker of therapeutic effectiveness; and as a biomarker of lifestyle and contextual effects related to cancer prevention, diagnosis, and progression of disease. In various embodiments, the methods provide means to decrease mortality rates, increase survival rates and decrease overall cancer associated health care expenditures, by improving detection, including early detection, detection of recurrences, measuring therapeutic effectiveness and monitoring modifiable lifestyle and contextual effects related to cancer linked to HPV infection. In one embodiment, the methods are used for triage and clinical management of HPV and SARS-CoV-2 co-infected patients in cervical cancer prevention clinics. In various embodiments, the methods are applied as high throughput detection/screening technology in a clinical setting. In various embodiments, the methods are used for staging a tumor, thus impacting clinical practice and population cancer incidence and prevalence rates.

In one embodiment, a sample is collected from the subject. The sample may be analyzed as described herein with respect to HPV, SARS CoV-2, biomarkers, or any combination thereof. If the analysis indicates that that subject has an increased risk of one or more of accelerating pre-malignant progression, accelerating progression, developing, or having cancer, the subject may be provided a follow-up for one or more of a biopsy or medical imaging (e.g., MRI, X-rays, ultrasound, CT scan, PET scan, endoscopy) of tissues associated with the risk.

In one example, the cancer associated with the risk is cervical cancer and the method includes performing or recommending a colposcopy. In one example, the cancer associated with risk is colon or rectal cancer and the method includes performing or recommending a colonoscopy. In one example, the cancer associated with the risk is lung, cervical, colorectal, pancreatic, or prostate cancer and the method includes performing or recommending a PET scan.

In one embodiment, the methods described herein or another method includes creating a personalized TCR repertoire for individual subjects from an initial premalignant or tumor sample biopsy to monitor the subject's tumor immune dynamics in biofluids such as: saliva for head and neck cancer; cervical epithelium scrapes, liquid cytology sample transport media (such as PreservCyt, ThinPrep and SurePath), and/or vaginal swabs (dry or in sample transport media) for cervical cancer; anal scrapes and/or swabs (dry or in sample transport media) for anal cancer; penile scrapes and/or swabs (dry or in sample transport media) for penile cancer; and blood and/or urine for head and neck, cervical, anal and penile cancer. In one example, creation or recommendation for the creation of the personalized TCR repertoire may be a step in a disclosed method upon a determination of a risk or particular level of risk. In one example, the personalized TCR repertoire may be created from the collected sample upon which a risk determination according to the present methods is determined or in a subsequently collected sample. In one configuration, the methods include periodically collecting and examining samples from the subject to detect a current TCR repertoire to monitor the subject's tumor immune dynamics in the biofluids.

In one embodiment, the methods include assigning a risk score with respect to the cancer risk, e.g., risk of accelerating pre-malignant progression, accelerating progression, developing, or having cancer. The risk score may be based on a presence, quantification, or characteristic with respect to one or more biomarkers in the presence of one or both of HPV or SARS CoV-2 virus in the sample or previously known. In a further example, a risk score may be based on a presence, quantification, or characteristic with respect to one or more biomarkers and a quantification or characteristic of one or both of HPV or SARS CoV-2 virus in the sample or previously known. In one example, a risk score is based on values established via correlation of presence, quantification, or characteristic with respect to one or more biomarkers and a quantification or characteristic of one or both of HPV or SARS CoV-2 virus in subjects having accelerated pre-malignant progression, accelerated progression of cancer, developing cancer, or having cancer, which may be identified relative to a population of normal subjects. For instance, results of the sample analysis may be compared to a correlation table or chart that assigns a risk score to the subject based on the correspondence of the results to results of subjects known to have the condition. In one example, risk scores may be associated with a priority or triage status of the subject. Risk scores may be associated with one or more procedures, courses of treatment (e.g., radiation, chemotherapy, chemoradiotherapy, or surgery such as ablative procedure, hysterectomy, or the like), follow-up procedures/tests (e.g., biopsy, excision, freezing (cryosurgery), laser, surgical removal, loop electrosurgical excision procedure (LEEP), cold knife conization, colposcopy, colonoscopy, imaging, biopsy), or recommendations thereof.

### Use Cases

The following examples are meant to provide nonlimiting example applications of various embodiments of the methods disclosed herein to aid the reader in better understanding the potential applications to which the methods may be applied.

Case 1: Early Detection of Cancer Linked to SARS-CoV-2. A biological sample, such as blood or saliva, is obtained from a subject with a history of SARS-CoV-2 infection. The sample is analyzed for the presence of viral and host biomarkers associated with cancer development, such as viral RNA or proteins, and host factors indicative of a host response to viral infection or cancer progression. If the biomarkers are detected at levels above predetermined thresholds, the subject may be considered at an increased risk for developing cancer linked to SARS-CoV-2 and may be recommended for further diagnostic evaluation or monitoring.

Case 2: Monitoring of Therapeutic Effectiveness in HPV-Associated Cancer. A subject diagnosed with cervical cancer linked to HPV infection undergoes chemotherapy and radiation therapy. Periodically, biological samples, such as blood or tissue biopsies, are obtained from the subject and analyzed for the presence of viral and host biomarkers. Changes in the levels of these biomarkers over time are correlated with the subject's response to therapy. If the biomarker levels decrease, the therapy may be considered effective, while if the biomarker levels remain elevated or increase, alternative treatment strategies may be explored.

Case 3: Relapse Monitoring of SARS-CoV-2-Associated Cancer. A subject previously treated for lung cancer linked to SARS-CoV-2 infection undergoes regular monitoring for cancer relapse. Blood samples are collected periodically and analyzed for the presence of viral and host biomarkers indicative of cancer recurrence. If the biomarker levels rise above predetermined thresholds, the subject may be recommended for further diagnostic evaluation to confirm cancer relapse and initiate appropriate treatment.

Case 4: Early Detection of HPV-Associated Cancer. A biological sample, such as cervical swab or saliva, is obtained from a subject at risk for HPV-associated cancer. The sample is analyzed for the presence of viral and host biomarkers associated with cancer development, such as HPV viral DNA or proteins, and host factors indicative of a host response to viral infection or cancer progression. If the biomarkers are detected at levels above predetermined thresholds, the subject may be considered at an increased risk for developing HPV-associated cancer and may be recommended for further diagnostic evaluation or monitoring.

Case 5: Integration with Imaging Techniques for Comprehensive Cancer Evaluation. In this embodiment, the method for detecting and monitoring viral and host biomarkers associated with cancer linked to SARS-CoV-2 and HPV is integrated with imaging techniques such as computed tomography (CT), magnetic resonance imaging (MRI), or positron emission tomography (PET) scans. The combined approach provides a comprehensive evaluation of the subject's cancer status, therapeutic response, and risk of relapse. The method allows for a more accurate and complete understanding of the subject's cancer, leading to improved treatment decision-making and better patient outcomes.

Case 6: Personalized Treatment Decisions Based on Biomarker Profiles. In this embodiment, the method for detecting and monitoring viral and host biomarkers associated with cancer linked to SARS-CoV-2 and HPV is used to guide personalized treatment decisions. By evaluating the biomarker profiles of individual subjects, healthcare providers can determine the most appropriate therapeutic interventions, taking into consideration the subject's unique cancer characteristics and risk factors. This personalized approach to treatment can lead to improved patient outcomes and reduced side effects.

Case 7: Monitoring Cancer Recurrence in HPV-Associated Cancer Survivors. In this example, a subject who has previously undergone treatment for HPV-associated cancer, such as cervical or oropharyngeal cancer, is monitored for cancer recurrence using the method of the present disclosure. Periodic biological samples are obtained from the subject and analyzed for the presence of viral and host biomarkers. If the biomarker levels rise above predetermined thresholds, the subject may be recommended for further diagnostic evaluation to confirm cancer recurrence and initiate appropriate treatment.

### EXPERIMENTAL EXAMPLES

The following nonlimiting experimental examples evidence various concepts disclosed herein.

### Example 1: SARS-CoV-2 Nucleic Acids in Cervical Liquid Cytology Specimens

A proof-of-principle study was performed to ascertain the presence of SARS-CoV-2 nucleic acids in cervical liquid cytology specimens, which had been tested for HPV.

Automated RNA extraction was performed using the KingFisher™ Flex Magnetic Particle Processor with 96 Deep-Well Head and the MagMAX™ Viral/Pathogen Nucleic Acid isolation Kit (Cat #A42352) or MagMAX™ Viral/Pathogen II Nucleic Acid Isolation Kit (Cat #A48383) with a sample input volume of 200 μL.

Briefly, 4 KingFisher™ Deep well 96 Plates (Cat #A48305) were prepared and labeled: “Wash 1” (Wash buffer). “Wash 2” (80% Ethanol). “Elution solution” and “Sample plate”. To each well of the “Sample plate”, 5 μL of Proteinase K, 200 μL of each sample was added, and 200 μL of nuclease-free water was added to the negative control well. Binding Bead Mix, previously prepared. was gently mixed five times, and 275 μL was added to each sample and the negative control well. Then, 5 μL of MS2 Phage control was added to each well. The MVP_2Wash_200_Flex program was used on the KingFisher™ Flex Magnetic Particle

Processor with 96 Deep-Well Head (Cat #5400630). After the run was completed, the “Elution Plate” was removed from the instrument and covered with MicroAmp™ Clear Adhesive Film (Cat #4306311). The samples were eluted in 50 μL of Elution Solution, placed on ice for immediate use in real-time RT-PCR assay. The purified nucleic acid was reverse transcribed into cDNA and amplified using the TaqPath™ RT-PCR COVID-19 Kit. To prepare the reaction mix. the following components were combined adequate for the number of samples to be tested, in addition to a positive control and a negative control: 6.25 μL of TaqPath™ 1-Step Multiplex Master Mix (No ROX™) (4×), 1.25 μL of COVID-19 Real-Time PCR Assay Multiplex, 7.50 μL of nuclease-free water for a total reaction mix volume of 15.0 μL. Then, we added either 10 μL of purified sample RNA (from RNA extraction), 10 μL of Purified Negative Control, or 2 μL of Positive Control (0.25 copies/μL of TaqPath™ COVID-19 Control) up to 25 μL of total volume to each well of the reaction plate. We performed the RT-PCR assay using the Applied Biosystems 7500 Fast Dx Real-Time PCR Instrument, and the SDS Software v1.4.1. with the following settings: Assay: Standard Curve (Absolute Quantitation), Run mode: Standard 7500, Passive reference: None, and Sample volume: 25 μL. The data was analyzed. interpreted and exported as .csv files using Applied Biosystems COVID-19 Interpretive Software (version 1.3). R (version 4.0.3) was used for biostatistics analyses.

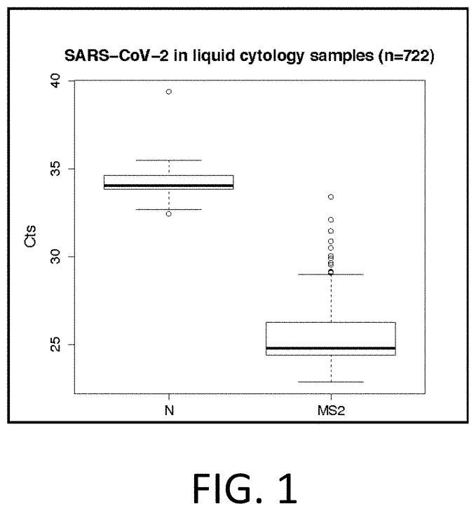

As shown in FIG. 1, the median of the SARS-CoV-2 nucleic acid Cycle Threshold (Ct) value was (min-max) for cases and 3.64 (2.86-4.13) for controls. The standard deviation for the global genomic DNA methylation index was 0.42 for cases and 0.46 for controls and the interquartile range was 1.14 for cases and 1.27 for controls.

### Example 2: Correlation Between Biomarkers of Premalignant Lesions and the Presence of SARS-CoV-2 Nucleic Acids in Cervical Epithelium Cells from Human Papilloma Virus Positive (HPV+) Subjects

Two competing hypotheses surmise how SARS-CoV-2 may impact premalignant progression of cervical epithelium from Low Squamous Intraepithelial Lesions (LSIL) to Cervical Intraepithelial Neoplasia (CIN) grades 1-3: (1) SARS-CoV-2 could directly infect cervical epithelium resulting in adverse effects and disease progression, and (2) COVID-19 indirectly impacts cervical dysplasia due to an exhausted immune system. Consequently, immune pressure on cervical tissue under SARS-CoV-2-infection is reduced enabling rapid progression of cervical dysplasia. We have previously shown that the CervicalMethDx test can provide a CIN grade 2-3 risk score, by assessing promoter DNA methylation by quantitative Real Time Methylation Specific PCR (qMSP) in a panel of three human genes (ZNF516 (e.g., NCBI Gene ID 9658), FKBP6 (e.g., NCBI Gene ID 8468), and INTS1 (e.g., NCBI Gene ID 26173)). Guerrero-Preston R, et al., Molecular Triage of Premalignant Lesions in Liquid-Based Cervical Cytology and Circulating Cell-Free DNA from Urine, Using a Panel of Methylated Human Papilloma Virus, and Host Genes. Cancer Prev Res (Phila). 2016 December; 9(12):915-924.

In this example, we evidence a correlation between biomarkers of premalignant lesions and the presence of SARS-CoV-2 nucleic acids in cervical epithelium cells from Human Papilloma Virus positive (HPV+) women using the CervicalMethDx test and the TaqPath COVID-19 Combo Kit (ThermoFisher) on discarded HPV+ cervical epithelium liquid cytology samples (n=696) in PreservCyt or SurePath media processed by clinical laboratories in Puerto Rico, from June 4 to Aug. 31, 2020. The TaqPath COVID-19 Combo kit is designed to coamplify sections of three SARS-CoV-2 viral genes: Nucleocapsid (N), Open Reading Frame lab (ORFlab), and Spike (S).

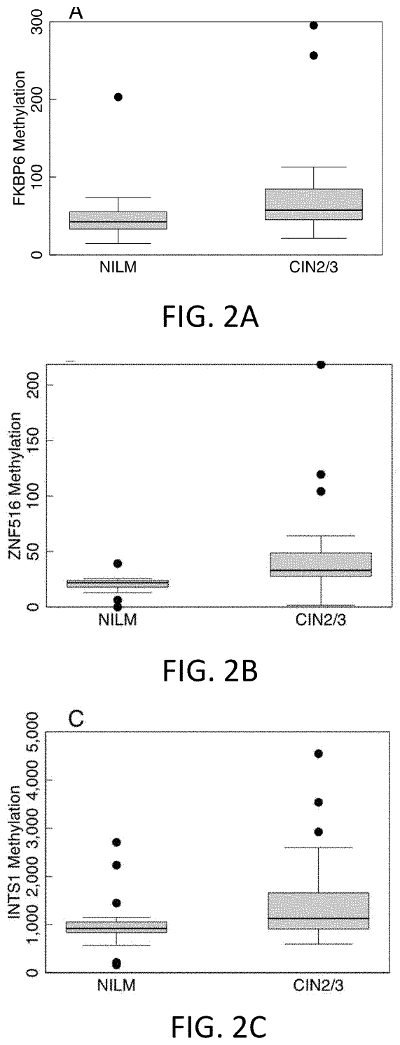

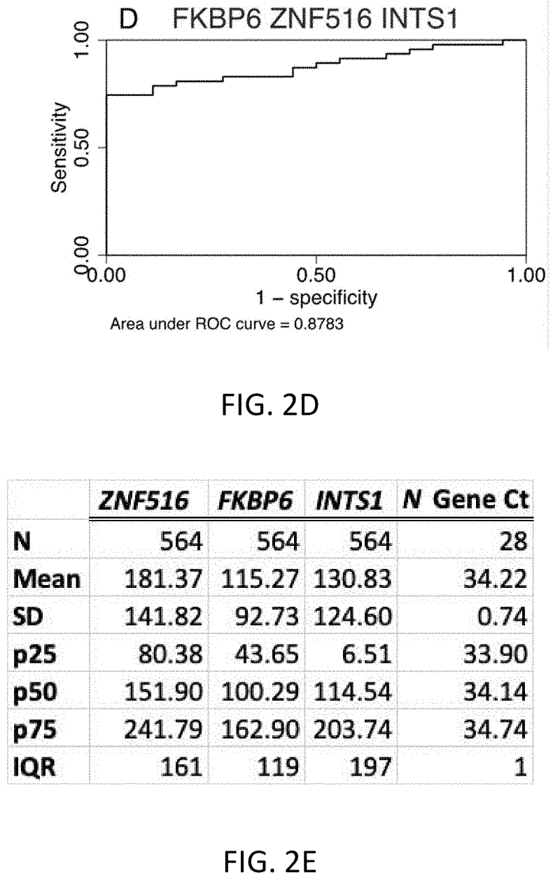

In this application, the CervicalMethDx test was able to correctly classify 86% of the discarded liquid cytology clinical samples from HPV-positive women when comparing DNA methylation in CIN2/CIN3 samples (n=47) to samples with a cervical pathology diagnosis of No Intraepithelial Lesions or Malignancy (n=18), with 78% Sensitivity, 94% Specificity, Area Under the Curve (AUC) of 0.88, and 98% positive predictive value. FIGS. 2A-2C show DNA methylation in liquid cytology samples from women with a pathology diagnosis with No Intraepithelial Lesions or Malignancy (NILM) and Cervical Intraepithelial Neoplasia Grade 2 or Grade 3 lesions (CIN 2/3) pathology diagnosis for FKBP6 (FIG. 2A); ZNF516 (FIG. 2B); and INTS) (FIG. 2C). Receiver Operator Characteristics (ROC) curve for panel of FKBP6, ZNF516, and INTS1 methylation in liquid cytology samples from women with NILM and CIN 2-3 pathology diagnosis are shown in FIG. 2D. N amplification was found in 5% of the samples with a Cycle Threshold (Ct) median of 34.1 and a range from 32.4 to 35.8 (FIG. 2E).

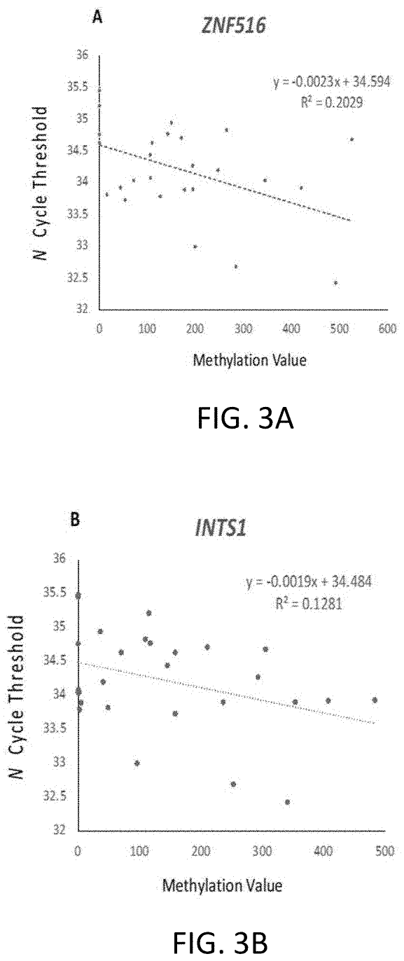

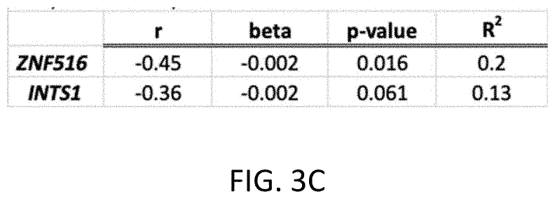

Linear relationship between the ZNF516 promoter DNA methylation and N Cycle Threshold (Ct) values in liquid cytology samples is shown in FIG. 3A. Linear relationship between INTS1 promoter DNA methylation and N Cycle Threshold (Ct) values in liquid cytology samples is shown in FIG. 3B. A statistically significant inverse pairwise correlation is established between ZNF516 methylation and N Ct values (−0.45; p=0.016) and a marginally significant correlation is established between INTS1 methylation and N Ct values (−0.36; p=0.061), as shown in FIG. 3C.

These data evidence a link between promoter DNA methylation of genes associated with CIN grade 2-3 risk and SARS-CoV-2 viral nucleic acids in cervical epithelium cells. In various embodiments, DNA methylation biomarkers, combined with clinical, cellular, and genetic factors, provide a useful tool for triage and clinical management of HPV and SARS-CoV-2 co-infected patients in cervical cancer prevention clinics.

### Example 3. Saliva and Biofluids as Surrogates for Diagnostic Immune-Profiling of Tumors, for Early Cancer Detection and Follow-Up of HPV Related Tumors in SARS-COV-2 Positive Patients or Patients with Long COVID, Who are Also HPV Positive

In this example, DNA samples were submitted for analysis of T-Cell receptor (TCR) quantity and clonality by TCR sequencing (TCR-seq) via enrichment of the human TCRB locus using Human-TCRB-PD4bx or Digital PCR as previously described (doi: 10.1158/1940-6207.CAPR-17-0356). CDR3 sequences were downloaded from Adaptive ImmuneAnalyzer. For each sequence, variable (V)-diversity (D)-joining (J) region genes were extracted and translated to amino acid on the CDR3 region. Paired Tumor, Lymphocytes and Saliva samples were submitted for shallow sequencing survey depth and deep assay depth. In order to better understand the TCR repertoire, the number of unique immune receptor clonotypes were assessed by examining their relative abundance, repertoire overlap, clonotype tracking and sequence length distribution in HPV positive and HPV negative samples, in SARS-CoV-2 positive and SARS-CoV-2 negative samples. Correlations and linear relationships were examined between known Immune system amino acid sequences, including but not limited to CDR3.aa, CD8+ and CD4+, HPV, SARS-CoV2 and other virus, such as CytoMegaloVirus, Epstein Barr Virus, Hepatitis B virus and Hepatitis C virus. CDR3.aa, CD8+ and CD4+ sequences annotated to HPV in HPV positive samples but annotated to other common viruses (CMV, EBV). This study demonstrates a robust TCR repertoire in saliva that corresponds at least partially to the clones observed in tumor and circulating lymphocytes. In addition, single-cell transcriptomes may be used to detect signatures of CD8+ and CD4+ neoantigen-reactive tumor-infiltrating Lymphocytes in paired saliva and tumor samples in addition to the viral or tumor-associated antigens present in bulk assays. These analyses may be performed in cancer-free individuals, as well as patients with HPV related premalignant lesions, and head neck, cervical, anal, and penile cancer.

### Example 4: T-Cell Receptors Quantity and Clonality as Immunoprevention Biomonitors in HPV Related Oropharyngeal Cancer and Premalignant Cervical Lesions

The concept of immunosurveillance, is based on the hypothesis that both the innate and adaptive immune systems provide an immunosurveillance function, which inherently identifies and eliminates aberrant cells, including tumor cells and builds a durable specific defense against them. This hypothesis also explains how tumors are able to escape from the antitumor immune response. An increased understanding of immunosurveillance mechanisms has laid the groundwork for the emerging field of cancer immunoprevention, which refers to the modulation of the host immune response to control the initiation or development of cancer. Cancer immunoprevention tools can be designed for use at different stages of oncogenesis, use of the HPV vaccine for cervical cancer prevention before the development of immune tolerance; using reverse immunology approaches to engineer patient-specific vaccines against predicted noepitopes during the latency period after initiation; or using the HER-2/neu (E75) vaccine to prevent breast cancer recurrence in high-risk patients.

T cell infiltration of solid tumors is associated with favorable patient outcomes. Infiltrating T lymphocytes (cytotoxic T cells, T helper 1 (TH1) cells, and memory T cells) are frequently found in malignant tumors and are suggestive of a host cancer immune response. The presence and quantity of tumor-infiltrating lymphocytes (TILs) correlate with increased patient survival, indicating that this immune signature could be an efficient biomarker of risk in the clinic. TILS can also be used for immunosurveillance purposes as TILs have been identified as a localized response in mucosal lesions to systemic therapeutic vaccination in the cervical cancer setting. TILs are also more frequently seen in patients with positive clinical response to antibodies that block immune inhibitory pathways. The US Food and Drug Administration (FDA) approved immunotherapy with antibodies that block CTLA-4 and the programmed cell death protein 1 (PD-1) for the treatment of advanced melanoma and non-small cell lung cancer (NSCLC). Identifying biomarkers that can predict response to check point inhibitors is critical to maximizing the benefit of these agents. PD-1 expression has been identified as a marker that can predict a positive response to anti-PD-1 immunotherapy. T-cell receptor clonality and quantity can also predict response to anti-PD-1 immunotherapy. However, it is not well understood what mechanisms determine the presence or absence of TILs in the tumor microenvironment. Tumor heterogeneity among cancer patients may be due to variability in germ-line genetic landscapes, somatic mutations in tumor cells and environmental differences.

Pathogen related tumors represent 20% of most solid tumors. High risk Human Papilloma Virus (hrHPV) is the etiological factor of cervical oropharyngeal, anal, and penile cancer. It is not known if infection and eventual insertion of hrHPV in the human genome is associated with T-cell receptor clonality and quantity in the tumor microenvironment. We set out to examine the association between T-cell receptor clonality and quantity and hrHPV status in head and neck cancer and premalignant cervical cancer samples.

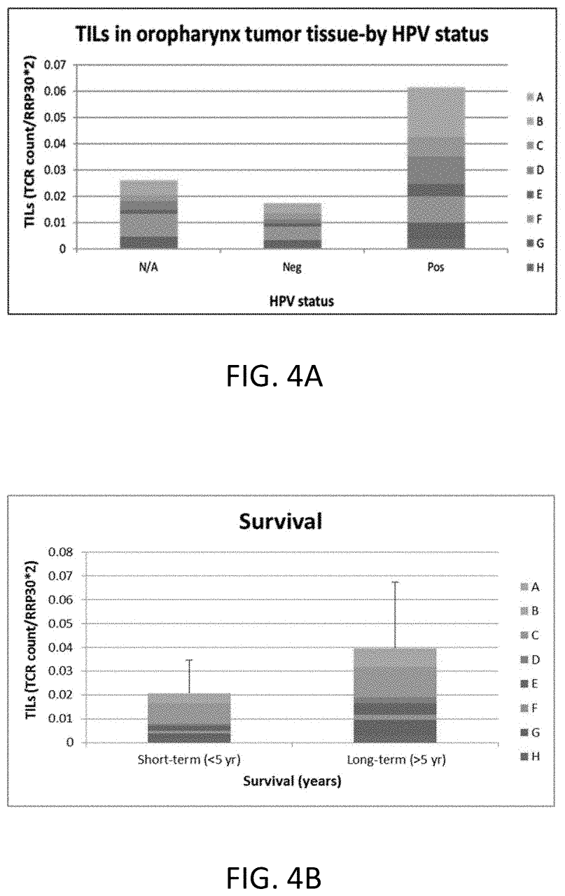

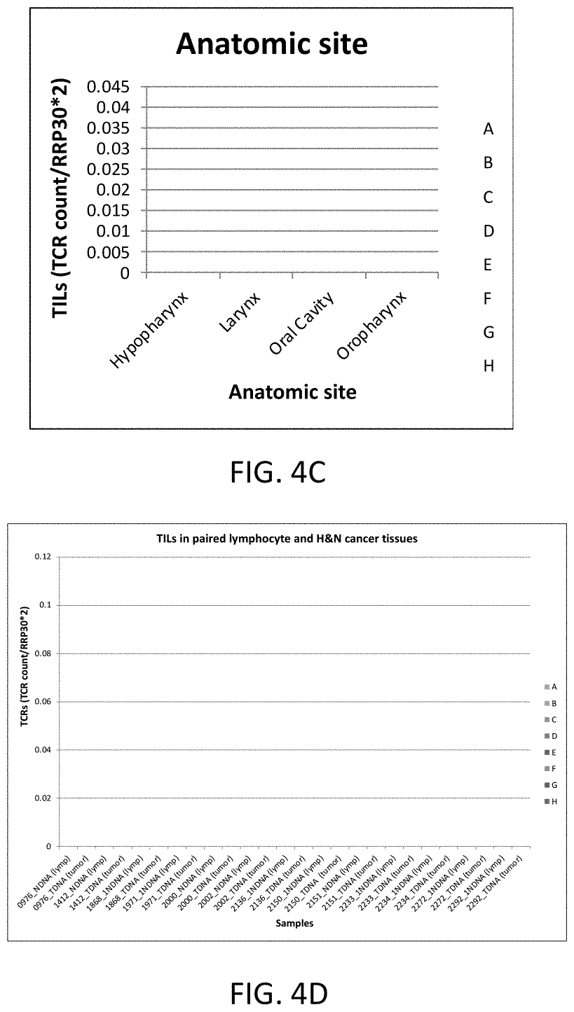

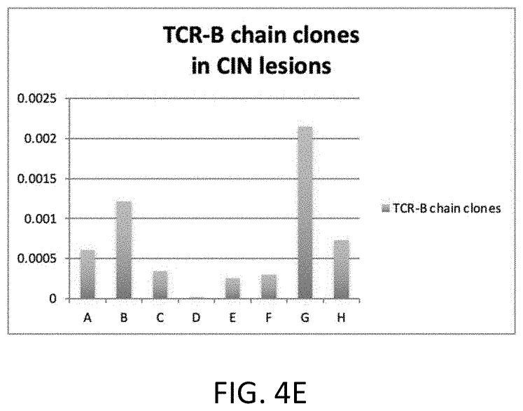

As shown in FIG. 4A, T-cell receptor (TCR) quantity and clonality in Tumor Infiltrating Lymphocytes (TILs) is higher in tumor tissue from oropharyngeal cancer patients positive to oncogenic HPV+ when compared with tumor tissue from HPV− oropharyngeal patients (p<0.05). Thus, TCR quantity and clonality in TILs

As shown in FIG. 4B, higher TCR quantity and clonality correlates with increased survival in head and neck cancer tissue samples (p<0.05). Thus, TCR quantity and clonality level may be used as a biomarker for cancer progression with respect to survival in tissue samples with respect to head and neck cancer.

FIG. 4C illustrates that TCR quantity and clonality differs by anatomic site in head and neck cancer patients while FIG. 4D shows tumor infiltrating lymphocytes in paired lymphocyte and Head and neck cancer tissues. As shown in FIG. 4E, T-cell receptor beta (TCR-b) chain clones' quantity differs in cervical intraepithelial Grade 1 and Grade 2 (CIN1/CIN2) lesions, with the G clone is the most abundant followed by the B and H clones.

Further to the above, a personalized TCR repertoire for individual patients may be created from an initial premalignant or tumor sample biopsy to monitor their tumor immune dynamics in biofluids such as: saliva for head and neck cancer; cervical epithelium scrapes, liquid cytology sample transport media (such as PreservCyt, ThinPrep and SurePath), and/or vaginal swabs (dry or in sample transport media) for cervical cancer; anal scrapes and/or swabs (dry or in sample transport media) for anal cancer; penile scrapes and/or swabs (dry or in sample transport media) for penile cancer; and blood and/or urine for head and neck, cervical, anal and penile cancer.

The present description includes various lists of elements. It is to be appreciated that the lists are to be understood as disclosing each element alone or in any combination of the listed elements, which may include the exclusion of any of the listed elements. For example, a method may include analysis of a sample comprising or consisting of a saliva sample, urine sample, tissue sample, or any combination thereof. The present description also describes the sample may be one or more of or selected from a saliva sample, urine sample, or tissue sample. Thus, the present description discloses a method that analyzes a salvia sample alone, a urine sample alone, tissue sample alone, a saliva sample and a urine sample, a saliva sample, and a tissue sample, as well as a urine sample and a tissue sample. The present description also discloses that tissue samples may include tissues, biofluids, or both from various locations. Thus, the present description describes methods in which any combination of disclosed samples of tissues, biofluids, or both, from any disclosed location, which may include multiple types of samples from different locations, are utilized. The present description also describes various categories of biomarkers including gene specific DNA methylation levels, whole genome DNA methylation levels, host RNA expression levels, T-Cell receptor amount or clonality, B-Cell receptor amount or clonality, differential microbiome characteristics with respect to enrichment, depletion, diversity, and presence relative to other bacteria for which the methods analyze the samples to detect. Thus, the present description discloses a method that includes detection of each biomarker category alone or with any combination of the other categories. The present description also discloses that the methods may comprise or consist of utilizing multiple biomarkers within the same category, e.g., detection of differential methylation of multiple gene specific promoter regions, or detection of microbiome differential including multiple species. Thus, the present description discloses a method that includes detection of a single biomarker in a single biomarker category, a single biomarker from each of two or more biomarker categories, detection of a single biomarker from a first biomarker category and two or more biomarkers from one or more additional biomarker categories. Any of these methods may also include any combination of sample type, sample location, or cancer, including multiples. Any of these methods may also include detection of one or more of presence, quantity, expression of SARS-CoV-2 and/or HPV DNA or RNA. The relevant analysis of SARS-CoV-2 and/or HPV nucleic acid may be performed on the same or different sample as one, more, or all the relevant analyses with respect to the biomarker detection. Thus, a first sample may be analyzed with respect to a first biomarker and one or more analyses with respect to SARS-CoV-2 and a second sample may be analyzed with respect to a second biomarker, wherein the first and second biomarkers correspond to identification of risk associated with the same cancer. As another example, a first sample may be analyzed with respect to a first biomarker in a first biomarker category and one or more analyses with respect to SARS-CoV-2 and a second sample may be analyzed with respect to a second biomarker in a second biomarker category, wherein the first and second biomarkers correspond to identification of risk associated with the same cancer. As another example, a first sample may be analyzed with respect to a first biomarker in a first biomarker category and one or more analyses with respect to SARS-CoV-2 and a second sample may be analyzed with respect to a second biomarker in a second biomarker category and one or more analyses with respect to SARS-CoV-2, wherein the first and second biomarkers correspond to identification of risk associated with different cancers. Any of these methods may also include detecting an increased risk of accelerated pre-malignant progression, development of cancer, having cancer, or cancer progression from the analysis of the same.

The techniques described herein generally utilize those known in the art unless described otherwise. Information relating to genes and genomes may be found at the NIH genetic sequence database GenBank at https://www.ncbi.nlm.nih.gov/genbank/. Non-limiting information regarding molecular and genetic techniques may be found in Pal, A., PROTOCOLS IN ADVANCED GENOMICS AND ALLIED TECHNIQUES, Springer US, (November 2021); O'Brien, W. (ed.), PRINCIPLES AND TECHNIQUES OF BIOCHEMISTRY AND MOLECULAR BIOLOGY, Syrawood Publishing House (March 2022); Hanns-Georg, K, et al., Whole genome sequencing (WGS), whole exome sequencing (WES) and clinical exome sequencing (CES) in patient care. J. Lab. Med. 38 (4) (2014) 221-230; Choi, M., et al., Genetic diagnosis by whole exome capture and massively parallel DNA sequencing, Proc. Natl. Acad. Sci. 106 (45) (2009) 19096-19101; Rother, K I, et al., Influence of DNA sequence and methylation status on bisulflte conversion of cytosine residues, Anal. Biochem. 231 (1) (1995) 263-265; Guan, W (Ed.), EPIGENOME-WIDE ASSOCIATION STUDIES, METHODS AND PROTOCOLS, Springer US (May 2022); Hatada, I, et al. (Ed.), EPIGENOMICS, METHODS AND PROTOCOLS, Springer US (September 2022); Ono, M (Ed.), Regulatory T-Cells, Methods and Protocols, Springer US (September 2022); Michels, K B (Ed.), EPIGENETIC EPIDEMIOLOGY, Springer International Publishing (April 2022); Deep, G (Ed.), CANCER BIOMARKERS, METHODS AND PROTOCOLS, Springer US (January 2022). The following documents include additional background information and techniques and are also incorporated herein by reference: U.S. Pat. No. 10,428,391, issued Oct. 10, 2019; Guerrero-Preston, R, et al., Key tumor suppressor genes inactivated by “greater promoter” methylation and somatic mutations in head and neck cancer, Epigenetics 9:7, 1031-1046; July 2014); Guerrero-Preston, R. NID2 and HOXA9 Promoter Hypermethylation as Biomarkers for Prevention and Early Detection in Oral Cavity Squamous Cell Carcinoma Tissues and Saliva, Cancer Prev Res (Phila) (2011) 4 (7): 1061-1072; Guerrero-Preston, R, et al., High-resolution microbiome profiling uncovers Fusobacterium nucleatum, Lactobacillus gasseri/johnsonii, and Lactobacillus vaginalis associated to oral and oropharyngeal cancer in saliva from HPV positive and HPV negative patients treated with surgery and chemo-radiation, Oncotarget, 2017, Vol. 8, (No. 67), pp: 110931-110948; Kerr A R. The oral microbiome and cancer. J Dent Hyg. 2015; 89: 20-3; Michaud D S, et al., Microbiota, oral microbiome, and pancreatic cancer. Cancer J. 2014; 20: 203-6; Narayanan V, et al., Human fecal microbiome-based biomarkersfor colorectal cancer. Cancer Prev Res (Phila). 2014; 7: 1108-11; Narayanan V, et al., Human fecal microbiome-based biomarkers for colorectal cancer. Cancer Prev Res (Phila). 2014; 7: 1108-11; Zambirinis C P, et al., Pancreatic cancer, inflammation, and microbiome. Cancer J. 2014; 20: 195-202; Seki E. Microbiome-obesity-liver cancer interaction: senescence of hepatic stellate cells and bile acids play new roles. Gastroenterology. 2014; 146: 860-1; Cook M B, et al., Serum pepsinogens and Helicobacter pylori in relation to the risk of esophageal squamous cell carcinoma in the alpha-tocopherol, beta-carotene cancer prevention study. Cancer Epidemiol Biomarkers Prev. 2010; 19: 1966-75; Shaw R. The epigenetics of oral cancer. Int J Oral Maxillofac Surg. 2006 February; 35(2):101-8; Righini C A, et al., Tumor-speciflc methylation in saliva: a promising biomarker for early detection of head and neck cancer recurrence. Clin Cancer Res 2007; 13:1179-85; Viet C T, et al., Methylation array analysis of preoperative and postoperative saliva DNA in oral cancer patients. Cancer Epidemiol Biomarkers Prev 2008; 17:3603-11.

All publications mentioned herein are incorporated herein by reference to disclose and describe the methods and/or materials to which they relate in the present description. The publications discussed herein are provided solely for their disclosure prior to the filing date of the present application. Nothing herein is to be construed as an admission that the present invention is not entitled to antedate such publication by virtue of prior invention.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not. For example, in an aspect, a disclosed method can optionally comprise one or more additional steps, such as, for example, repeating an administering step or altering an administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

The present disclosure may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

What is claimed is:

1. A method for detection of cancer risk mediated by SARS-CoV-2 and oncogenic Human Papilloma Virus (HPV), the method comprising:
quantifying, in a sample isolated from a subject, SARS-CoV-2 nucleic acids;
analyzing a sample isolated from a subject for one or more host biomarkers associated with a risk of a cancer; and
comparing an amount of SARS-CoV-2 nucleic acids in a sample to the presence of HPV DNA or RNA, wherein, if the value of SARS-CoV-2 nucleic acids in the sample is independently high or high relative to a quantification of the HPV DNA or RNA and the one or more biomarkers are detected in HPV positive subjects then the subject has an increased risk of premalignant progression associated with the cancer, having the cancer, or progression of the cancer.

2. The method of claim 1, wherein the sample from which SARS-CoV-2 nucleic acids are quantified corresponds to a same tissue or biofluid from which the presence of HPV was quantified.

3. The method of claim 1, wherein the HPV has been previously quantified.

4. The method of claim 1, wherein comparing the amount of SARS-CoV-2 nucleic acids in the sample to the presence of HPV DNA or RNA comprises comparing the levels of SARS-CoV-2 gene expression in a sample to the presence of HPV DNA or RNA, and wherein the value comprises an expression value of the SARS-CoV-2 gene.

5. The method of claim 1, wherein the sample from which SARS-CoV-2 nucleic acids are quantified corresponds to a same tissue or biofluid that is analyzed for at least one of the one or more biomarkers.

6. The method of claim 1, wherein the one or more biomarkers comprise an expression value of one or more host genes in the sample that correlates (positively or negatively) in HPV and SARS-CoV-2 positive subjects.

7. The method of claim 1, wherein the one or more biomarkers comprise a level of gene specific DNA methylation of one or more host genes and a host RNA expression level corresponding to the one or more genes that positively or negatively correlate with HPV infected subjects also infected with SARS-CoV-2 or having long COVID.

8. The method of claim 1, wherein said method further comprises the following steps:
isolating a sample from said subject, wherein said sample comprises genomic DNA;
performing sodium bisulfite conversion of genomic DNA to differentiate and detect unmethylated versus methylated cytosines associated with premalignancy and/or malignancy in patients coinfected with HPV and SARS-CoV-2 or having long COVID;
using massively parallel sequencing methods or methylation arrays to reveal the methylation status at individual cytosine level associated with premalignancy and/or malignancy in patients coinfected with HPV and SARS-CoV-2 or having long COVID;
comparing the levels of whole genome DNA methylation with SARS-CoV-2 gene expression or amplification and the presence of HPV DNA or RNA (predetermined level), wherein the HPV has been previously quantified, whereby if the levels of whole genome DNA methylation amplification in the sample is low in SARS-CoV-2 and HPV positive subjects, or patients having long COVID, then the subject has an increased risk of having cancer;
using quantitative methylation specific PCR (qMSP) to identify Differentially Methylated regions associated with premalignancy and/or malignancy in patients coinfected with HPV and SARS-CoV-2 or patients having long COVID;
comparing the levels of gene specific DNA methylation and host RNA expression levels, wherein RNA can be, mRNA, microRNA, or long-non-coding RNA, with the presence of SARS-CoV-2 and HPV DNA or RNA (predetermined level), wherein HPV and SRAS-CoV-2 have been previously quantified, whereby if concordant levels of host DNA methylation and RNA expression levels in the sample are correlated (positively or negatively) with SARS-CoV-2 and HPV subjects, or patients having long COVID, then the subject has an increased risk of having cancer; and
comparing the amount and clonality of T-Cell receptors and B-Cell receptors in the samples with gene specific DNA methylation level, whereby, if gene Specific DNA methylation is inversely correlated to T-Cell receptors and/or B-Cell receptors amount or clonality in SARS-CoV-2 and HPV positive subjects, or patients having long COVID, then the subject has an increased risk of having cancer.

9. The method of claim 1, wherein the one or more biomarkers comprise a differential promoter methylation of one or more host genes relative to corresponding samples of unaffected subjects.

10. The method of claim 1, wherein the one or more biomarkers comprises an amount and/or clonality of T-Cell and/or B-Cell receptors in the sample, and wherein the method further includes analyzing the sample to quantifying an amount and/or clonality of T-cell and/or B-cell receptors in the sample.

11. The method of claim 1, wherein the one or more biomarkers comprises a microbiota differential relative to corresponding samples of unaffected subjects, and wherein the method further comprises analyzing the sample for presence of a microbiota differential.

12. The method of claim 1, wherein the one or more biomarkers comprise a predetermined low level of whole host genome DNA methylation in the sample relative to corresponding samples of unaffected subjects.

13. The method of claim 1, wherein the one or more biomarkers comprise an inverse correlation between a gene specific DNA methylation with respect to one or more host genes and T-cell receptors and/or B-cell receptors amount or clonality.

14. The method of claim 1, wherein the sample comprises a first tissue or biofluid and a second tissue or biofluid.

15. The method of claim 1, further comprising isolating the sample from the subject.

16. The method of claim 1, wherein the sample comprises a cervical liquid cytology sample, saliva sample, urine sample, cervical smear, vaginal lavage fluid sample, anal smear, stool sample, tumor sample, tissue sample, or any combination thereof.

17. The method of claim 1, wherein the cancer is, oral cancer, tongue cancer, oropharyngeal cancer, anal cancer, penile cancer, vulvar cancer, or vaginal cancer.

18. A method for detection of cancer risk mediated by SARS-CoV-2 and Human Papilloma Virus, the method comprising:
determining a subject has an increased risk of accelerated premalignant progression associated with a cancer, having the cancer, or progression of the cancer if the subject is HPV positive and a biospecimen sample isolated from the subject corresponding to a tissue or biofluid secreted or contacting tissue associated with the cancer if an amount of SARS-CoV-2 nucleic acids in the sample is independently high or high relative to an amount of HPV DNA or RNA in the sample or a previous sample a biospecimen sample isolated from the subject corresponding to a tissue or biofluid secreted or contacting tissue associated with the cancer.
